SEQUENCE LISTING (34 sequences)

SEQ ID NO: 1 - RNA, 21 nt, Artificial Sequence
  Description of Artificial Sequence Synthetic oligonucleotide
uagcaccaucugaaaucgguu

SEQ ID NO: 2 - RNA, 23 nt, Artificial Sequence
  Description of Artificial Sequence Synthetic oligonucleotide
uagcaccauuugaaaucaguguu

SEQ ID NO: 3 - RNA, 23 nt, Artificial Sequence
  Description of Artificial Sequence Synthetic oligonucleotide
uagcaccauuugaaaucaguguu

SEQ ID NO: 4 - RNA, 21 nt, Artificial Sequence
  Description of Artificial Sequence Synthetic oligonucleotide
uagcaccauuugaaaucgguu

SEQ ID NO: 5 - DNA, 27 bp, Artificial Sequence
  Description of Artificial Sequence Synthetic primer
ccgctcgagttggtttggccctttatc

SEQ ID NO: 6 - DNA, 26 bp, Artificial Sequence
  Description of Artificial Sequence Synthetic primer
cggaattcccaccctgcttacctctg

SEQ ID NO: 7 - DNA, 20 bp, Artificial Sequence
  Description of Artificial Sequence Synthetic primer
atgccattccgaatctcagc

SEQ ID NO: 8 - DNA, 20 bp, Artificial Sequence
  Description of Artificial Sequence Synthetic primer
catctggcgatgtaatgatg

SEQ ID NO: 9 - DNA, 20 bp, Artificial Sequence
  Description of Artificial Sequence Synthetic primer
tgatggtggcttcactgtgt

SEQ ID NO: 10 - DNA, 20 bp, Artificial Sequence
  Description of Artificial Sequence Synthetic primer
ttccatctctggtgaagctg

SEQ ID NO: 11 - DNA, 20 bp, Artificial Sequence
  Description of Artificial Sequence Synthetic primer
gtgatccataatctggagga

SEQ ID NO: 12 - DNA, 20 bp, Artificial Sequence
  Description of Artificial Sequence Synthetic primer
ccgaatgacacactctcttc

SEQ ID NO: 13 - DNA, 22 bp, Artificial Sequence
  Description of Artificial Sequence Synthetic primer
ggtctttacggatgtcaacgtc

SEQ ID NO: 14 - DNA, 30 bp, Artificial Sequence
  Description of Artificial Sequence Synthetic primer
ctagtctagaggggcaatatgtttaacaag

SEQ ID NO: 15 - DNA, 27 bp, Artificial Sequence
  Description of Artificial Sequence Synthetic primer
gctctagactggctggactaaacctct

SEQ ID NO: 16 - DNA, 30 bp, Artificial Sequence
  Description of Artificial Sequence Synthetic primer
ggactagtgatggtggtcgtcatatttatt

SEQ ID NO: 17 - DNA, 31 bp, Artificial Sequence
  Description of Artificial Sequence Synthetic primer
gctctagataacatacacgtcttactaaata

SEQ ID NO: 18 - DNA, 32 bp, Artificial Sequence
  Description of Artificial Sequence Synthetic primer
ggactagtgcatccaagatggttatttattta

SEQ ID NO: 19 - DNA, 15 bp, Artificial Sequence
  Description of Artificial Sequence Synthetic oligonucleotide
atttcagatggtgct

SEQ ID NO: 20 - DNA, 20 bp, Artificial Sequence
  Description of Artificial Sequence Synthetic primer
catcagagagcaactgatcc

SEQ ID NO: 21 - DNA, 20 bp, Artificial Sequence
  Description of Artificial Sequence Synthetic primer
gctacgttttttacctccac

SEQ ID NO: 22 - DNA, 20 bp, Artificial Sequence
  Description of Artificial Sequence Synthetic primer
gtgagtctttggaggtcctt

SEQ ID NO: 23 - DNA, 20 bp, Artificial Sequence
  Description of Artificial Sequence Synthetic primer
tccacagacatccatagagc

SEQ ID NO: 24 - DNA, 20 bp, Artificial Sequence
  Description of Artificial Sequence Synthetic primer
tctgctcaaaaacgccatct

SEQ ID NO: 25 - DNA, 20 bp, Artificial Sequence
  Description of Artificial Sequence Synthetic primer
caccatttggcactgctttc

SEQ ID NO: 26 - DNA, 21 bp, Artificial Sequence
  Description of Artificial Sequence Synthetic primer
gctacagcttcaccaccacag

SEQ ID NO: 27 - DNA, 30 bp, Artificial Sequence
  Description of Artificial Sequence Synthetic primer
ctagtctagatgcttgtgtttgtaagtctg

SEQ ID NO: 28 - DNA, 29 bp, Artificial Sequence
  Description of Artificial Sequence Synthetic primer
gctctagagcattgcacagtttatttcca

SEQ ID NO: 29 - DNA, 28 bp, Artificial Sequence
  Description of Artificial Sequence Synthetic primer
ggactagtgaagatctgtgtggtctctc

SEQ ID NO: 30 - DNA, 26 bp, Artificial Sequence
  Description of Artificial Sequence Synthetic primer
gctctagatgcttcggtgggtgggat

SEQ ID NO: 31 - DNA, 26 bp, Artificial Sequence
  Description of Artificial Sequence Synthetic primer
ggactagtccctccaccagctcatgg

SEQ ID NO: 32 - DNA, 20 bp, Mus sp.
ctgtggtgctctgtggtgct

SEQ ID NO: 33 - DNA, 10 bp, Artificial Sequence
  Description of Artificial Sequence Synthetic oligonucleotide
ctgggtttcg

SEQ ID NO: 34 - DNA, 20 bp, Artificial Sequence
  Description of Artificial Sequence Synthetic oligonucleotide
ctgggtttcgctgggtttcg

(19) United States
(12) Patent Application Publication
(10) Pub. No.: US 2011/0178159 A1
(43) Pub. Date: Jul. 21, 2011

(54) INHIBITORY RNAS THAT REGULATE HEMATOPOIETIC CELLS

(76) Inventors: Hua Gu, New York, NY (US); Yoon-Chi Han, New York, NY (US); Christoph Park, Stanford, CA (US); Irving Weissman, Stanford, CA (US)

(21) Appl. No.: 12/991,656

(22) PCT Filed: May 8, 2009
(86) PCT No.: PCT/US09/43353

§ 371 Date: Mar. 25, 2011

Related U.S. Application Data
(60) Provisional application No. 61/051,626, filed on May 8, 2008.

(51) Int. Cl. A61K 31/7088; C12Q 1/68; C12N 5/071; C12N 5/0786; C12N 5/0787; A61P 35/00; A61P 35/02

(52) U.S. Cl. 514/44 A; 435/6.14; 435/375; 435/6.13

(57) ABSTRACT

Provided are compositions and methods for preventing, treating, ameliorating or diagnosing conditions or diseases involving a myeloid cell proliferation disorder. Such compositions and methods target miRNA function myeloproliferative diseases. More particularly, such compositions and methods target miR-29a function in myeloid cell proliferation disorders. Also provided are methods for diagnosing risk or presence of a myeloid cell proliferation disorder in a subject.

This application claims priority to U.S. application Ser. No. 61/051,626, filed on May 8, 2008.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Hematopoiesis is a step-wise development of multiple lineages of hematopoietic cells starting from long-term hematopoietic stem cells (HSCs). A general scheme of hematopoiesis depicts that HSCs first develop into multipotential progenitors (MPP) which can then give rise to common lympoid progenitors (CLP) and common myeloid progenitors (CMP) with a more restricted lineage potential. These progenitors can further differentiate into multi-lineages of T, B, and myeloid cells in the bone marrow (BM) and thymus. See Rosenbauer and Tenen, Nature Reviews Immunology, 2007 for a review of hematopoietic linage diversification. Although both HSCs and hematopoietic progenitors are proliferating cells and have the potential to develop into multiple lineages of hematopoietic cells, HSCs are the only cell population that is capable of self-renewal, a critical function to maintain the life-long hematopoiesis.

Leukemia is a malignant cancer of the bone marrow and blood. It is characterized by the uncontrolled growth of blood cells. The common types of leukemia are divided into four categories: acute or chronic myelogenous, involving the myeloid elements of the bone marrow (white cells, red cells, megakaryocytes) and acute or chronic lymphocytic, involving the cells of the lymphoid lineage. The pathogenicity of myelogenous leukemias is associated with inappropriate or uncontrolled proliferation of myeloid lineage cells. For example, chronic myelogenous leukemia (CML) is characterized by increased and unregulated growth of predominantly myeloid cells in the bone marrow, and accumulation of those cells in the blood.

Like HSCs, leukemia cells, such as those found in acute myeloid leukemia (AML), also possess a hierarchical developmental structure, with leukemia stem cells (LSCs), or leukemia initiating cells, sit at the top of the developmental cascade that may give rise to more differentiated, developmentally heterogeneous multiple myeloid lineage of cells. While many leukemias contain a high proportion of proliferating leukemia cells, LSCs are the only leukemia cell population that may self-renew and capable of transfer the diseases. Thus, understanding the mechanisms that control the self-renewal capability of HSCs and LSCs is of fundamentally importance to the prevention and treatment of hematopoietic disorders and malignancy.

There is a great need for agents and compounds allowing the treatment and prevention of leukemia or of differentiation and development disorders of blood cells and blood progenitor cells. This invention addresses this need.

MicroRNA (miRNA) of ˜22 nucleotides are a class of small regulators that control development and metabolism of various organisms. Although microRNAs have been shown to be aberrantly expressed in numerous human cancers, such studies have not demonstrated a direct role for microRNAs in tumorigenesis. MicroRNAs (miRNAs) regulate numerous cellular processes including proliferation, differentiation, and apoptosis (Chang and Mendell, 2007; He and Hannon, 2004). They regulate gene expression by repressing protein translation form coding mRNAs or promoting degradation of the target mRNAs. Given that the miRNA regulation usually depends on the match of its seed sequences of about 6-8 nucleotides with their target sequences usually located within the 3′UTR of the target mRNA, one miRNA may simultaneously regulates multiple targets in the same cells.

miRNAs are differentially expressed in different lineages of hematopoietic cells. In the hematopoietic system, miRNAs have been shown to regulate lineage commitment and mature effector cell function (Chen and Lodish, 2005; Garzon and Croce, 2008). miRNAs can play an important role in tumor genesis, evidenced by frequent deletion of chromosomal regions containing miRNAs or miRNA clusters, and altered patterns of miRNA expression in various tumors. Over-expression of miR-155 in early B cells leads to polyclonal expansion of pro-B compartment. Retroviral expression of miR-155 in mouse hematopoietic system results in myeloproliferative disorder. While these data all show that miR-155 can have role of in hematopoietic cell proliferation, they do not show that it can influence the function of HSCs and LSCs.

### SUMMARY OF THE INVENTION

We show that miRNA miR-29a is highly expressed in HSCs and down-regulated in hematopoietic progenitors. Over-expression of miR-29a in the hematopoietic system can cause stem cell-like self-renewal capability in hematopoietic progeintors, biased lineage development toward myeloid lineages and development of a myeloproliferative disorder (MPD) that progresses to AML. Over-expression of miR-29a can convert short-lived myeloid progenitors into self-renewal populations. miR-29a is also highly expressed in myeloid leukemia cells and leukemia stem cells (LSCs). The inventors show that miR-29a miRNA can convert myeloid progenitors into LSCs for the development and progression of AML, and that miR-29a can regulate hematopoietic stem cells (HSC), committed progenitors, and leukemia stem cells (LSC). In addition, miR-29a levels can serve as a novel diagnostic marker in the diagnosis of human myeloid leukemia and other diseases of the human hematopoietic stem cells (HSC). Moreover, inhibitors of miR-29a can be used to treat AML and CML.

Therefore, the present invention relates to compositions and methods for preventing, treating, ameliorating or diagnosing conditions or diseases involving a myeloid cell proliferation disorder. The present invention also relates to such compositions and methods that target miRNA function in such diseases. More particularly, the present invention relates to such compositions and methods that target miR-29a function in diseases involving white blood cell proliferation. In another aspect, the invention relates to methods for diagnosing whether of not a subject has, or is at risk of having a myeloid cell proliferation disorder.

In a particular embodiment, the invention provides a method for treating a miR-29a-induced myeloproliferative disorder in a subject, the method comprising administering to the subject an miRNA inhibitory nucleic acid specific to miR-29a.

For purposes of this invention, “treating” includes preventing progression of an existing disease, delaying onset and/or severity of disease, and ameliorating or reducing the severity, frequency, duration, etc., of one or more symptoms of disease.

A “myeloproliferative disorder” (MPD) is a condition characterized by abnormal red blood cell, white blood cell, and/or platelet growth, predominantly in the bone marrow, but sometimes in the liver and spleen as well. MPDs include acute myelogenous leukemia (AML), chronic myelogenous leukemia (CML), chronic neutrophilic leukemia (CNL), chronic eosinophilic leukemia/hypereosinophilic syndrome (CEL), polycythemia vera, agnogenic myeloid metaplasia, essential thrombocytosis, primary thrombocythemia, primary or idiopathic myelofibrosis (myelosclerosis), and myelodysplastic syndrome.

A miR-29a-induced MPD is an MPD wherein HSCs or myeloid cells of the patient or subject show increased expression of miR-29a over that of a normal or control individual not having an MPD. A miR-29a-induced MPD is characterized by or associated with higher-than-normal levels of miR-29a in HSCs or myeloid cells of the patient or subject having the miR-29a-induced MPD.

In one aspect, the myeloproliferative disorder is a leukemia, in particular, AML, CML, CNL, or CEL. In one embodiment, the leukemia is selected from the group consisting of AML and CML.

The miRNA inhibitory nucleic acid is preferably an antagomir or an miRNA sponge. In one embodiment, the antagomir has a sequence of SEQ ID NO: 19.

In another aspect, the invention provides a method for diagnosing a miR-29a-induced myeloproliferative disorder in a patient, the method comprising comparing miR-29 expression in a myeloid cell from the patient with miR-29a expression in a myeloid cell from a control subject, wherein greater miR-29 expression in the patient indicates a a miR-29a-induced myeloproliferative disorder. The control subject is an individual who does not have a miR-29a-induced MPD.

In one aspect, the myeloproliferative disorder is a leukemia, in particular, AML, CML, CNL, or CEL. In one embodiment, the leukemia is selected from the group consisting of AML and CML.

The invention further provides a method for reducing miR-29a-induced proliferation of a myeloid cell comprising administering to the myeloid cell an miRNA inhibitory nucleic acid specific to miR-29a.

In one embodiment, the myeloid cell is selected from the group consisting of a monocyte, a granulocyte, a mast cell, a basophil, and a megakaryocyte.

The miRNA inhibitory nucleic acid is preferably an antagomir or an miRNA sponge. In one embodiment, the antagomir has a sequence of SEQ ID NO: 19.

The invention further provides a method of identifying a compound for reducing miR-29a-induced myeloid cell proliferation, the method comprising:
(a) administering to a non-human test mammal a candidate compound, wherein the test mammal comprises myeloid cells overexpressing miR-29a; and
(b) comparing miR-29a expression in myeloid cells from the test mammal with miR-29a expression in myeloid cells from a non-human control mammal, wherein the control mammal overexpresses miR-29a;

wherein reduced miR-29a expression in the test mammal indicates that the candidate compound is a compound for reducing myeloid cell proliferation.

In one embodiment, the non-human mammal has a myeloproliferative disorder as defined herein.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

As would be apparent to one of ordinary skill in the art, any method or composition described herein can be implemented with respect to any other method or composition described herein.

### BRIEF DESCRIPTION OF THE DRAWINGS

FIG. 1. miR-29a is expressed at high levels in hematopoietic stem cells (HSC) as well as human AML. FIG. 1A: Heatmap of miR-29a expression. Expression was normalized against sno-R2 and data is presented as a z-score. FIG. 1B: Absolute Ct values for miR-29a expression in FACS sorted normal human BM populations and human AML shows that miR-29a is expressed at highest levels in HSC. FIG. 1C: Absolute Ct values for miR-29a expression in FACS purified mouse HSC/progenitors shows a dramatic decrease in miR-29a expression in multipotent progenitors (MPP) (MPPA=Lin⁻Kit⁺Sca⁺CD34⁺Flk2⁻, MPPB=Lin⁻Kit⁺Sca⁺CD34⁺Flk2⁺).

FIG. 2. Ectopic expression of mir-29a in mouse hsc/progenitors induces myeloproliferative disease in primary chimeras. FIG. 2A: Flow cytometric analysis of the bone marrow and spleen of miR-29a transduced, long-term engrafted (3-5 months) primary chimeras reveals that miR-29a induces monocytic/granulocytic hyperplasia in both compartments. FIG. 2B: Primary miR-29a chimeric mice exhibit signs of myeloproliferative disease including increased splenic extramedullary hematopoiesis (shown by arrows) as well as granulocytic and megakaryocytic hyperplasia in both the spleen and bone marrow. Cytopsin preparations of the bone marrow were stained with Wright-Giemsa to reveal a predominance of maturing granulocytic precursors and near-absence of erythroid precursors in miR-29a mice relative to normal controls (bottom). FIG. 2C: The myeloproliferative phenotype is associated with changes in the immature hematopoietic compartment, manifested by increased proportions of Lin-Kit-cells and phenotypic HSC, as well as the relative expansion of normal myeloid progenitors populations. These findings are representative of at least 5 independent experiments.

FIG. 3. miR-29a expression alters the proliferative, differentiation, and self-renewal capacity of hematopoietic progenitors at the level of the multipotent progenitor. FIG. 3A: Clone-sorted miR-29a MPP (sorted either as Lin⁻Kit⁺Sca⁺CD34⁺FLK2⁻ or Lin⁻ Kit⁺Sca⁺CD34⁺SLAM⁻) show a higher proliferative capacity in liquid culture compared to WT MPP, but this difference is not observed in CMP or GMP. FIG. 3B: Lineage potential was assessed based on evaluation of cytospin preparations from clone-sorted MPP in vitro liquid cultures, demonstrating that miR-29a promotes monocytic differentiation and reduces megakaryocytic differentiation. FIG. 3C: Statistics of flow cytometric evaluation of the peripheral blood of mice long-term engrafted (>16 weeks post-transplant) with sorted MPP from miR-29a MPD mice reveals a relative myeloid hyperplasia with statistically significant differences in myeloid and lymphoid output from miR-29a MPP-derived (identified as GFP⁺ cells) and the control recipient's HSC/progenitors (identified as GFP⁻ cells). FIG. 3D: Flow cytometric analysis of hematopoietic cells in sorted CMP and GMP reconstituted mice. Control cells are wild type (GFP⁻) cells. Left panel: anti-CD16 and anti-CD34 staining of gated donor derived (GFP⁺) Lin-c-Kit⁺Sca-1⁻ bone marrow (BM) cells. Middle panel: anti-Mac-1 and anti-Gr-1 staining of donor derived (GFP⁺) bone marrow (BM) cells. Right panel: anti-TCR and anti-B220 staining of donor-derived (GFP⁺) splenic cells. Results are representatives of more than three mice.

FIG. 4. miR-29a myeloproliferative disease evolves to an acute myeloid leukemia that phenotypically resembles a myeloid progenitor and contains an LSC population. FIG. 4A: Secondary transplant recipients become morbid ˜3-4 months post transplant and exhibit splenomegaly and hepatomegaly at necropsy (top left). Histologic sections of the bone marrow (top right) and spleen (bottom left) show effacement of normal architecture by sheets of myeloid blasts. Wright-Giemsa stain of a cytospin preparation of total splenocytes shows a marked expansion of immature blasts that exhibit myeloid and monocytic cytologic features with the presence of frequent cytoplasmic granules. FIG. 4B: Flow cytometric phenotypic analysis of BM cells reveals marked expansion of granulocyte-macrophage progenitor-like (GMP-L) cells (Lin⁻Kit⁺Sca⁻CD34⁺CD16/32⁺) cells. FIG. 4C: Transplantation with as few as 20 sorted GMP-L cells is sufficient to transfer leukemia to unirradiated secondary recipients. The transplanted leukemia resembles that of the primary leukemia, with increased numbers of GMP-L as well as more differentiated leukemic cells.

FIG. 5. miR-29a Over-expression expedites cell cycle progression. FIG. 5A: Mac-1⁺Gr-1⁺ splenic granulocytes were evaluated for proliferation status by DAPI staining during the MPD phase of disease, revealing that miR-29a granulocytes contain a higher fraction of proliferating granulocytes. BM B cells were used as positive control for DAPI staining FIG. 5B: Analysis of cell cycle status by BrdU incorporation in 293T cells reveals increased numbers of miR-29a 293T cells in S/G2 consistent with the observed increased rate of proliferation as assessed by cell counts. FIG. 5C and FIG. 5D: Increased numbers of miR-29a 293T cells in S/G2 phase corresponds to increased rate of proliferation as assessed by cell counts.

FIG. 6. Evaluation of potential gene targets reveals that Hbp1 is a bona fide target of miR-29a. FIG. 6A: Western blot analysis for HBP1 protein expression in sorted primary granulocytes from control and miR-29a-expressing mice reveals that HBP1 expression is diminished in miR-29a cells. FIG. 6B: Semi-quantitative RT-PCR for additional predicted target genes of miR-29a identifies several additional potential targets. The sloped line indicates decreasing amounts of input RNA for the reactions. FIG. 6C: Luciferase reporter assays reveal that both predicted miR-29a binding sites in the 3′UTR of Hbp1 mediate miR-29a′s inhibitory effect on gene expression. FIG. 6D: Western blot showing a decreased protein level of Hbp1 in Gr-1+/Mac-1+ cells overexpressing miR-29a, but not Pten.

FIG. 7. Generation of miR-29a chimeras. FIG. 7A: Schematic representation of the MSCV-based miR-29a expression construct used in expression studies. The construct was a gift from C. Z. Chen. FIG. 7B: Northern blot analysis of 293T cells transduced with the miR-29a retrovirus demonstrates that miR-29a that is expressed and properly processed to its mature, 21 nt length. FIG. 7C: Time course of primary chimera generation and evaluation. Following treatment of wild type (CD45.1) mice with 5-FU, the bone marrow was harvested and transduced with the miR-29a-expressing retrovirus. Following transduction, bulk bone marrow cells were transplanted into lethally irradiated, congenic (CD45.2) recipients. Chimerism was evaluated beginning at 6-8 weeks and monitored on a monthly basis.

FIG. 8. Progressive myeloid expansion in the peripheral blood of engrafted, primary miR-29a chimeric mice. Flow cytometric analysis was performed on peripheral blood leukocytes using Mac-1⁺ at the indicated times.

FIG. 9. Expansion of myeloid cells and decrease in mature B cells in primary chimeric mice is seen 2-3 months post-transplant. FIG. 9A: The bone marrow of miR-29a recipient mice exhibits a marked increase in GFP⁺Mac-1⁺ myeloid cells and near-absence of GFP⁺B220⁺ B cells. This effect is specific to hematopoietic cells derived from miR-29a expressing progenitors, as indicated by the presence of GFP positivity. FIG. 9B: The spleen of secondary recipient mice shows similar features to the bone marrow, with near-absence of B220⁺ B cells.

FIG. 10. Altered myeloid progenitor profiles in miR-29a induced myeloproliferative disease. Some mice showed expansion of a novel myeloid progenitor (Lin⁻Kit⁺Sca⁻CD16/32⁺CD34⁻), while others showed a relative expansion of immunophenotypic MEP. In both cases, notice the expansion of Lin⁻Kit⁻ cells, consistent with left-shifted myeloid maturation.

FIG. 11. Summary of HSC/progenitor cell composition in miR-29a chimeric mice exhibiting myeloproliferative disease. Control group includes WT (untransduced) and Emp retrovirus-transduced mice. miR-29a mice included those exhibiting signs of myeloproliferative disease, defined as splenomegaly with expanded immature myeloid compartment and no increase in blasts, as assessed by morphology. Data were analyzed by a two-tailed T-test, and statistically significant differences are denoted with an asterisk.

FIG. 12. miR-29a expression promotes myeloid differentiation while inducing decreased commitment to the B cell lineage. Flow cytometric evaluation of the bone marrow of primary chimeric mice demonstrates reduced numbers of progenitor B-cells (total B220⁺IgM⁻ cells), although the ratios of maturing B cell progenitor subsets is relatively unaltered.

FIG. 13. Southern blot analysis of sequential transplants of miR-29a transduced BM. Genomic DNA was prepared from total BM cells from lines 1 and 2. Both lines represent miR-29a transduced cells in which primary chimeras showed MPD features and in which the MPD evolve to AML during passage 2. Note the stability of bands in line 1, indicating the absence of selection for a particular clone. Line 2 shows possible slight differences in a subline between the 2nd and 3rd passages, suggesting the possibility of an oligoclonal disease undergoing selection pressures. The probe used was against GFP.

FIG. 14. miR-29a expression induces differential expression of numerous mRNAs. FIG. 14A: mRNA microarray analysis of Mac-1⁺Gr-1⁺ cells purified from the spleens of stably engrafted miR-29a primary chimeras and empty vector control mice reveals numerous genes that are differentially expressed. FIG. 14B: Semi-quantitative PCR performed on total RNA purified from sorted miR-29a expressing Mac-1⁺Gr-1⁺ cells confirms decreased mRNA expression for Dnmt31 and increased expression of Hdac6 and Lrrk1. Bak1, Dpp3, and Casp7 are not significantly decreased.

FIG. 15. Hbp1 knockdown in hematopoietic progenitors does not alter ratios of myeloid progenitors or mature hematopoietic cells in the bone marrow of engrafted mice. FIG. 15A: Western blot analysis of 3DO cells expressing Hbp1 shRNA constructs reveals that construct Hbp1-2 efficiently decreases protein expression. Construct Hbp1-2 was used for all subsequent in vivo experiments. FIG. 15B: Myeloid progenitor and mature myeloid cell composition in BM were evaluated by flow cytometry in primary chimeric mice stably engrafted with Hbp1shRNA GFP⁺ cells.

FIG. 16. Hbp1 knockdown does not alter mature myeloid or lymphoid output in engrafted mice. The percentage of Hbp1 shRNA expressing GFP⁺ granulocytes and lymphoid cells in SP were evaluated in the spleen using the indicated markers.

FIG. 17. Differential Expression of miR-29a in Hematopoietic Lineage Cells. Microarray and Northern blot analyses of miR-29a expression. miR-29a exhibited different patterns of expression in different lineages of hematopoietic cells, with miR-29a expressed at a high level in lymphocytes but moderately detectable in myeloid cells (FIG. 17A). Microarray (FIG. 17B) and Northern blot (FIG. 17C) analyses of miR-29a expression.

FIG. 18. The miR29 family of microRNAs and Polycistron. FIG. 18A: The mir-29 family is polycistronic. miR-29a and miR-29b-1 are located in mouse chromosome 6A3 and human chromosome 7q32.2 which are approximately 250 bp apart. Likewise, miR-29c and miR-29b-2 are located in mouse chromosome 1H6 and human chromosome 1q32.2 which are approximately 500 bp apart. FIG. 18B: Although all members of the miR-29 family share the same “seed” sequence (positions 2-8 from the 5′ end of the miRNA, their flanking sequences vary. The sequences are miR-29a UAGCACCAUCUGAAAUCGGUU (SEQ ID NO: 1); miR-29b-1 UAGCACCAUUUGAAAUCAGUGUU (SEQ ID NO: 2); miR29b-2 UAGCACCAUUUGAAAUCAGUGUU (SEQ ID NO: 3); and miR-29c UAGCACCAUUUGAAAUCGGUU (SEQ ID NO: 4).

FIG. 19. Ectopic Expression of miR-29a Impairs B Lymphopoiesis. Flow cytometric analysis of B and T cells in the bone marrow and spleen. Shown are Mac-1 and B220 staining of total bone marrow cells, and B220 and CD3 staining of total spleen cells from the BM chimeras. Window (far right) shows a significantly altered ratio of Mac-1 to B220 ratio in the bone marrow, and B220 to CD3 ratio in spleen in miR-29a expressing population. The percentages of each cell population are indicated in the plots. BM: bone marrow; SP: spleen.

FIG. 20. Ectopic Expression of miR-29a Results in Myeloid Lineage Hyperplasia. miR-29a over-expression results in myeloid hyperplasia. FIG. 20A: miR-29a over-expressing BM chimera exhibited splenomagaly. FIG. 20B: Giemsa staining of splenic leukocytes revealed that miR-29a overexpressing BM chimeras possessed significantly more myeloid-granuloid cells of immature phenotypes

FIG. 21. Myeloid Hyperplasia of miR-29a Over-Expressing Cells in Spleen and Bone marrow. Increased number myeloid cells in miR-29a BM chimera mice. Spleen (FIG. 21A) and bone marrow (FIG. 21B) sections from empty vector (vector) or miR-29a (miR-29a) overexpressing virus infected BM chimeric mice were stained with H&E. Shown are 20× and 40× images.

FIG. 22. Expansion of miR-29a expressing myeloid cells in the recipient mice. Shown are the total numbers of GFP+ Mac-1+ cells before and post 150 days of transfer. Total spleen cells from either empty vector (white bar) or miR-29a overexpressing (hatched and shaded bars) bone marrow chimera was transferred to wildtype C57B1/6 mice. After 5 month, increased GFP+ Mac-1+ cells was detected only in mice receiving miR-29a overexpressing splenocytes, but not that receiving empty vector infected splenocytes.

FIG. 23. Biogenesis of microRNA (mir). Mature mir is generated by two processing events by Drasha in the nucleus and Dicer in the cytoplasm. Mature mir then can bind to its target mRNA for mRNA cleavage or translation repression.

FIG. 24. Ectopic expression of miR-29a results in myeloid lineage hyperproliferation. miR-29a over-expression results in myeloid hyperplasia. Top left: qRT-PCR of Emp and miR-29a BMR Gr-1+/Mac-1+ sorted cells showing overexpression of miR-29a. Right: Giemsa staining of splenic leukocytes revealed that miR-29a overexpressing BM chimeras possessed significantly more myeloid-granuloid cells of immature phenotypes. Bottom: BM B220+ B cells as a positive control for DAPI staining, miR-29a chimera Gr-1+/Mac-1+ cells are in cell cycle and proliferating in comparison with Emp chimera Gr-1+/Mac-1+.

FIG. 25. Emp and miR-29a Transfer. MicroRNA-29a myeloid cells are serially transplantable. To ensure that the expansion of myeloid cells are due to leukemia and not a polyclonal expansion, total splenocytes from emp and miR-29a were transferred to wildtype recipient mice. About 4 months after the transfer, the recipient mice were analyzed by flow cytometry. In contrast to emp control, miR-29a transferred mice had GFP+ which are all Gr-1+/Mac-1+ cells.

FIG. 26. miR-29a family expression in human HSC, AML and different progenitor cells. miR-29a is highly expressed in HSC compared to different progenitor cells. Like the HSC, AML patients also show high miR-29a expression. HSC and different progenitor cells were collected from normal patients. Mir-29 expression was detected using Taqman qRT-PCR.

FIG. 27. Increased GMP in miR-29a overexpressed BMR Spleen. Spleen GMP population is also greatly expanded in miR-29a BM chimera mice. BM cells were stained for GMP using Lin-Sca-1-c-Kit+ then CD16 and CD34 for different myeloid progenitors.

FIG. 28. mir-29a Over-expressing GMP aquired capability of self-renewal. To ensure that GMP has gained self-renewal capability and acting as leukemic stem cells, 20 GMP sorted cells of B6SJL(control since express CD45.1) and miR-29a chimera mice were transferred to wildtype recipients (CD45.2). About 1.5 months after the transfer, the recipient mice were analyzed by flow cytometry. GMP from the bone marrow and spleen cells show no CD45.1 cells in the control B6SJL while almost 100% of cells were GFP+ in miR-29a. miR-29a GMP cells are expanding in the recipient mice thus, show that it acquired capability of self-renewal.

FIG. 29. miR-29a over-expressing GMP retained capability of differentiation potential. To ensure that the expanding GMP cells from miR-29a are functionally the “true” GMP cells and give rise to granulocytes and macrophage, the bone marrow and spleen from the recipient mice were stained with Gr-1 and Mac-1. Majority of Gr-1+/Mac-1+ cells are GFP+ miR-29a expressing cells.

FIG. 30. Hbp1 (HMG-box protein 1). Domain organization of Hbp1. Repression domain n modulated by RB and p130, but also has independent intrinsic repression activity (Classon et al, 2001). Overexpression of Hbp1 results in inhibition of G1 and S-phase progression (Tevosian et al, 1997). A general suppressor of Wnt signaling-Cyclin D1 and c-Myc (Sampson et al, 2001). Consistently upregulated in differentiation and under conditions of cell cycle arrest in muscle, adipocyte, erythroid and other cell types (Yee et al, 1998). Hbp1 overexpression enhanced myeloid cell line K562 cell toward erythroid and megakaryocyte lineage (Yao et al, 2005). Hbp1 gene lines within the 7q31 region that is frequently deleted or translocated in breast cancer and AML (acute myeloid leukemia) (Zenklusen et al, 1994, Koike et al, 1999).

FIG. 31. Hbp1 is a direct target of miR-29a. As emp vector as normalization, full length 3′UTR of Hbp1 with both miR-29a binding sites present showed 40% decrease in luciferase activity while 1^{st} mutant has a little higher in activity. 2^{nd} mutant showed similar level as the full length and 1+2 mutant was comparable to the empty vector control. This suggests that Hbp1 is a direct target of miR-29a and that 1^{st} tentative miR-29a binding site seems to be important.

FIG. 32. Hbp1 and proliferation barriers in differentiation. Hbp1 is reported to bind to Rb and regulates cell cycle, inhibit cell proliferation (G1 to S phase progress), to allow cells to differentiate.

FIG. 33. Treatment of primary AML cells with miR-29a inhibitor. Stock LNA concentration was 10 mg/ml. Therefore, 1/100=0.1 mg/ml, 1/200=0.05 mg/ml, etc.

### DETAILED DESCRIPTION OF THE INVENTION

The present invention is related to the biology and function of microRNAs (miRNAs), a class of non-coding RNAs of about 22 nucleotides in length, in the process of hematopoiesis. miRNA cellular functions can be inhibited by specific inhibitory nucleic acids that competitively inhibit mRNA binding to miRNAs. In one aspect, the present invention relates to the use of miRNA inhibitors, such as antagomirs, in treating leukemic conditions. The present disclosure shows that antagomirs inhibitors of miR-29a are effective in the treatment of abnormal blood cell proliferation. miRNA inhibitory nucleic acids that inhibit miR-29a inhibit the proliferation of myeloid lineage cells associated with leukemias or pre-leukemic conditions, for example, blood disorders such as myelodysplastic or myeloproliferative syndromes.

A patient who has been diagnosed with a disorder characterized by unwanted miRNA expression (e.g., unwanted expression of miR-29a) can be treated by administration of an miRNA inhibitory nucleic acid described herein to block the negative effects of the miRNA, thereby alleviating the symptoms associated with the unwanted miRNA expression. Similarly, a human who has or is at risk for developing a disorder characterized by under expression of a gene that is regulated by an miRNA can be treated by the administration of an miRNA inhibitory nucleic acid that targets the miRNA. For example, a human diagnosed with Parkinson's disease. The patient can be administered an miRNA inhibitory nucleic acid that targets endogenous miR-29a, which binds Hbp1 RNA in vivo, presumably to downregulate translation of the Hbp1 mRNA and consequently downregulate Hbp1 protein levels.

Therefore, in one aspect, the invention relates to a method for treating or preventing a preleukemia stage in myeloid leukemogenesis by inhibiting self-renewal capability by myeloid progenitors by inhibiting mir-29a expression. Inhibition of miR-29 expression can be used for the treatment of human AML using primary human AML samples in a xenotransplantation setting. In another embodiment, inhibition of miR-29a expression can be used to treat a subject having an AML or a related diseases, either alone or in combination with one or more conventional treatment.

In yet another embodiment, inhibition of miR-29a expression can be used to treat a subject having a myeloid lineage cell proliferation-related disorder. In one embodiment, the myeloid lineage cell proliferation-related disorder is a leukemia. In another embodiment, the myeloid lineage cell proliferation-related disorder is a pre-leukemic condition. In still a further embodiment, the myeloid lineage cell proliferation-related disorder is a myeloid leukemia. In still a another embodiment, the myeloid lineage cell proliferation-related disorders is an acute myeloid leukemia, a chronic myeloid leukemia, a myelodysplastic syndrome, a myeloproliferative disease or any combination thereof.

The present methods can also be used to inhibit myeloid lineage cell proliferation which is non-pathogenic; i.e., myeloid lineage cell proliferation which results from normal processes in the subject (for example during infection). Thus, the invention provides a method for inhibiting non-pathogenic myeloid lineage cell proliferation.

In another aspect, the invention provides a method for diagnosing a a myeloid cell proliferation disorder, or a pre-leukemic condition. The amount of miR-29a in a myeloid cell, or a myeloid cell progenitor can be used microRNA as a marker for diagnosis of a myeloid cell proliferation disorder. The results described herein show that human hematopoietic stem cells and myeloid leukemia and leukemia stem cells express a high levels of miR-29a and expression of miR-29a alone in hematopoietic stem cells or myeloid lineage progenitors is sufficient to convert these stem cells or progenitors into myeloid leukemia stem cells, resulting in the development of acute myeloid leukemia. The expression of miR-29a in hematopoietic cells can be quantitatively determined by any method known in the art, including quantitative determination by PCR or DNA chip assay. In another embodiment, an siRNA chip-based assay may be used to diagnose human myeloid leukemia and as a therapeutic strategy to treat miR29a-related myeloid leukemia.

Diagnostic methods include detecting the amount of miR-29a expressed in a cell from a subject and comparing the amount to the amount of miR-29a expressed in a reference or control cell from a second subject not having a myeloid leukemogenesis, wherein a greater amount of miR-29a expressed in the cell from the first subject compared to the reference cell indicates that the first subject has a myeloid leukemogenesis. In one embodiment, the myeloid leukemogenesis is a preleukemia stage myeloid leukemogenesis.

In another embodiment, the invention provides methods to determine the level of miR-29a in hematopoietic cells, such as PCR, DNA and RNA microarrays to quantify miR-29a expression. In one embodiment, expression level of miR-29 can be used to screen one or more patients having or and risk of having a myeloid cell proliferation disorder. In one embodiment, upregulation of miR-29a in myeloid cells can be indicative of a pre-leukemic state of a myeloid cell proliferation disorder. In another embodiment, upregulation of miR-29a in myeloid cells can be indicative of a leukemic state of a myeloid cell proliferation disorder

In one aspect, the invention described herein relates to a method for determining differentiation of a hematopoietic lineage cell, the method comprising measuring the expression level of miR-29a. In one embodiment, the expression level of miR-29a can be used to differentiate a HSC from a committed myeloid or lymphoid progenitor. In another embodiment, he expression level of miR-29a can be used to differentiate a MPP from a committed myeloid or lymphoid progenitor.

In another aspect, the invention described herein relates to a method for affecting hematopoietic development by expressing miR-29a in a cell. In one embodiment, miR-29a can be overexpressed to altering myeloid progenitor composition. In another embodiment, miR-29a can be overexpressed to promote myeloid differentiation. In still a further embodiment, miR-29a can be overexpressed to promote MPP proliferation. In yet another embodiment, miR-29a can be overexpressed to establish a granulocyte/macrophage lineage bias among myeloid progenitors.

In yet another aspect, the invention described herein relates to a method for converting a non-self-renewing myeloid progenitor cells into a self-renewing cell by over-expressing miR-29a in the cell. In one embodiment, over-expression of miR-29a can induce aberrant self-renewal of CMP and GMP without impairing their normal ability to differentiate into monocytes or granulocytes. In another embodiment, over-expression of miR-29a can be used to cause aberrant acquisition of self-renewal capability by committed progenitors such as CMP and GMP.

In still another aspect, the invention described herein realtes to a method for prognosis of human AML and other diseases of HSC/progenitors, wherein the absence or presence of miR-29a can be used to evaluate for residual and/or recurrent AML. In one embodiment, the methods featured in the invention are useful for reducing the level of an endogenous miRNA (e.g., miR-29a) or pre-miRNA in a cell, e.g., in a cell of a subject, such as a human subject or mouse subject. Such methods include contacting the cell with an miRNA inhibitory nucleic acid, such as a single-stranded oligonucleotide agent, described herein for a time sufficient to allow uptake of the miRNA inhibitory nucleic acid into the cell.

In another aspect, the invention features a pharmaceutical composition including an miRNA inhibitory nucleic acid, such as a single-stranded oligonucleotide, and a pharmaceutically acceptable carrier. In a preferred embodiment, the miRNA inhibitory nucleic acid hybridizes under physiological or intracellular conditions to miR-29a.

The invention provides a method of inhibiting miRNA expression (e.g., miR-29a) or pre-miRNA expression in a cell. The method includes contacting the cell with an effective amount of an miRNA inhibitory nucleic acid, such as a single-stranded oligonucleotide, described herein, which is substantially complementary to the nucleotide sequence of the target miRNA or the target pre-miRNA. Such methods can be performed on a mammalian subject by administering to a subject one of the miRNA inhibitory nucleic acids as described herein or a pharmaceutical composition comprising such miRNA inhibitors. In a preferred aspect, the target miRNA differs by no more than one nucleotide from the sequence of any of SEQ ID NO: 1-4.

In another aspect, the invention features a method of increasing levels of an RNA or protein that are encoded by a gene whose expression is down-regulated by an miRNA, for example an endogenous miRNA, such as miR-29a. The method includes contacting the cell with an effective amount of an miRNA inhibitory nucleic acid, such as a single-stranded oligonucleotide, which is substantially complementary to the nucleotide sequence of the miRNA that binds to and effectively inhibits translation of the RNA transcribed from the gene. For example, the invention features a method of increasing Hbp1 protein levels in a cell. The methods include contacting the cell with an effective amount of an miRNA inhibitory nucleic acid described herein, which is substantially complementary to the nucleotide sequence of miR-29a. Hbp1 protein levels can be increased in a cell by contacting the cell with an effective amount of an isolated nucleic acid comprising a nucleotide sequence sufficiently complementary to a microRNA target sequence, wherein the target sequence differs by no more than 1 nucleotide from the sequence of any of SEQ ID NO: 1-4.

In still another aspect, the invention provides a method of reducing the amount of a microRNA, preferably miR-29a, in a cell comprising introducing an isolated nucleic acid into the cell, wherein the isolated nucleic acid comprises a sequence which is substantially complementary to a target miRNA sequence, and wherein the target miRNA sequence differs by no more than 1, 2, or 3 nucleotides from the sequence of any of SEQ ID NO: 1-4.

In still a further aspect, the invention provides a transgenic non-human mammal whose somatic and/or germ cells are transduced with a nucleic acid from about 50 nucleotides to about 250 nucleotides or about 110 to about 130 nucleotides in length, comprising a nucleotide sequence at least about 75%, 80%, 85%, 90%, 92%, 95%, 98% or 100% identical to the nucleotide sequence of any of SEQ ID NO: 1-4. The invention provides a transgenic non-human mammal whose somatic and/or germ cells are transduced with a nucleic acid sufficiently complementary to a microRNA target sequence, wherein the target sequence differs by no more than 1 nucleotide from the sequence of any of SEQ ID NO: 1-4. The nucleic acid is operably linked to a promoter. In one embodiment, the promoter is a constitutively active promoter or an inducible promoter. In another embodiment, the promoter is a cell specific promoter. In yet a further embodiment, the cell for which the promoter is specific is a hematopoietic cell, or any progenitor thereof.

In still a further aspect, the invention provides a method for determining whether a compound is capable of treating a myeloid lineage cell proliferation-related disorder in a transgenic non-human mammal whose somatic and germ cells comprise a nucleic acid segment encoding of any of SEQ ID NO: 1-4 operably linked to a promoter, the method comprising, (a) administering a test compound to the transgenic non-human mammal whose somatic and germ cells comprise a nucleic acid segment encoding of any of SEQ ID NO: 1-4 operably linked to a promoter (b) measuring progression of myeloid lineage cell proliferation-related disorder in the transgenic non-human mammal of (a); and (c) comparing the progression the myeloid lineage cell proliferation-related disorder measured in (b) to progression of a myeloid lineage cell proliferation-related disorder measured in a sibling of the transgenic non-human mammal, wherein the sibling was not administered the test compound, and wherein progression of the myeloid lineage cell proliferation-related disorder of the non-human mammal of (a) compared to (b) indicates the that the test compound is capable of treating a myeloid lineage cell proliferation-related disorder.

In still a further aspect, the invention provides a method for determining whether a test compound modulates intracellular levels a nucleic acid comprising any of the sequences shown in any of SEQ ID NOs: 1-4, the method comprising; a) contacting a cell expressing a nucleic acid comprising the sequence shown in any of SEQ ID NO: 1-4 with a test compound; b) determining an amount of the nucleic acid in the cells of step (a); and c) comparing the amount of the nucleic acid in (b) to the amount of the nucleic acid in a cell in the absence of the test compound, wherein an increase or decrease in the amount of the nucleic acid in (b) indicates that the test compound modulates the intracellular levels of the nucleic acid comprising the sequence shown in any of SEQ ID NO: 1-4

### DEFINITIONS

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “complimentary” as used herein refers to nucleotide sequences in which the bases of a first oligonucleotide or polynucleotide chain are able to form base pairs with a sequence of bases on another oligonucleotide or polynucleotide chain. The terms “sense” and “antisense” refer to complimentary strands of a nucleotide sequence, where the sense strand or coding strand has the same polarity as an mRNA transcript and the antisense strand or anticoding strand is the coding strand's compliment. The antisense strand is also referred to as the anticoding strand.

As used herein, “substantially complementary” refers to when the nucleotides of one strand of a nucleic acid pair with at least about 80% of the nucleotides of the other strand. In one embodiment, the complementarity is at least about 90%, at least about 95%, or least about 98%, or at least about 99%, or about 100% to the nucleotides of the other strand. The two or more strands of nucleic acid that hybridize can be, for instance, two strands of a DNA molecule or an oligonucleotide primer and a primer binding site on a single stranded nucleic acid, or two single-stranded portions of a single nucleic acid. Complementary nucleotides are, generally, A and T (or A and U), or C and G. It should be noted that for all nucleotide sequences provided herein, T can substitute for U and vice versa. In one embodiment, substantial complementary exists when an RNA or DNA strand will hybridize to its complement under selective hybridization conditions. Typically, selective hybridization will occur when there is at least about 65% complementarity over a stretch of at least 14 to 25 nucleotides, at least about 75% complementarity over a stretch of at least 14 to 25 nucleotides, or at least about 90% complementarity over a stretch of at least 14 to 25 nucleotides. See, M. Kanehisa Nucleic Acids Res. 12:203 (1984), incorporated herein by reference.

As used herein, “substantially single stranded” when used in reference to a nucleic acid molecule means that the nucleic acid molecule exists primarily as a single strand of nucleic acid. Single stranded RNA or DNA molecules are substantially single stranded when less than about 20% of the nucleotides in the RNA or DNA molecule are not hybridized to a nucleotide of another RNA or DNA molecule. In one embodiment, a nucleic acid is substantially single stranded when less than about 10%, less than about 7%, less than about 5%, less than about 2% or less than about 1% of the nucleotides in the molecule are not hybridized to a nucleotide of another RNA or DNA molecule.

### MicroRNAs

A variety of nucleic acid species are capable of modifying gene expression. These include antisense RNA, siRNA, microRNA, RNA and DNA aptamers, and decoy RNAs. MicroRNAs (miRNAs) are a class of 18-24 nt non-coding RNAs (ncRNAs) that are processed from hairpin precursors of 70 nt (pre-miRNA) which are derived from primary transcripts (pri-miRNA) through sequential cleavage by the RNAse III enzymes Drosha and Dicer.

An miRNA or pre-miRNA can be 18-100 nucleotides in length. Mature miRNAs can have a length of 19-30 nucleotides, 21-25 nucleotides, particularly 21, 22, 23, 24, or 25 nucleotides. MicroRNA precursors typically have a length of about 70-100 nucleotides and have a hairpin conformation. MicroRNAs are generated in vivo from pre-miRNAs by the enzymes Dicer and Drosha, which specifically process long pre-miRNA into functional miRNA. The miRNA-type oligonucleotide agents, or pre-miRNA-type oligonucleotide agents featured in the invention can be synthesized in vivo by a cell-based system or in vitro by chemical synthesis. MicroRNA-type oligonucleotide agents can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting.

miRNAs have roles in a variety of biological processes including developmental timing, differentiation, apoptosis, cell proliferation, organ development, and metabolism (Chang and Mendell, 2007; He and Hannon, 2004). They regulate gene expression by repressing protein translation form coding mRNAs or promoting degradation of the target mRNAs. Given that the miRNA regulation usually depends on the match of its seed sequences of about 6-8 nucleotides with their target sequences usually located within the 3′UTR of the target mRNA, one miRNA may simultaneously regulates multiple targets in the same cells.

miRNAs are differentially expressed in different lineages of hematopoietic cells. In the hematopoietic system, miRNAs have been shown to regulate lineage commitment and mature effector cell function (Chen and Lodish, 2005; Garzon and Croce, 2008). miRNAs can play an important role in tumor genesis, evidenced by frequent deletion of chromosomal regions containing miRNAs or miRNA clusters, and altered patterns of miRNA expression in various tumors. Over-expression of miR-155 in early B cells leads to polyclonal expansion of pro-B compartment. Retroviral expression of miR-155 in mouse hematopoietic system results in myeloproliferative disorder. While these data all show that miR-155 can have role of in hematopoietic cell proliferation, they do not show that it can influence the function of HSCs and LSCs.

The present invention provides specific compositions and methods that may be useful for treating or preventing the occurrence of a leukemia in e.g., a mammal, such as a human or a mouse. In one aspect, the present invention provides specific compositions and methods to reduce levels of the miRNA miR-29a for the treatment or prevention of a myeloid cell proliferation disorder.

As described herein, miRNA miR-29a is highly expressed in HSCs and down-regulated in hematopoietic progenitors. In one aspect, over-expression of miR-29a in hematopoietic system can cause biased lineage development toward myeloid lineages and development of MPD that progresses to AML. In another aspect, over-expression of miR-29a can convert short-lived myeloid progenitors into self-renewal populations. Given that miR-29a is also highly expression in some human AML, the results described herein show that miRNA can convert myeloid progenitors into LSCs for the development and progression of AML and that miR-29a can regulate hematopoietic stem cells (HSC), committed progenitors, and leukemia stem cells (LSC).

One basis for the invention disclosed herein is the discovery that miR-29a is expressed at high levels in human hematopoetic stem cells, myeloid leukemia and leukemia stem cells. The results described herein further show that miR-29a is highly expressed in normal HSC and downregulated in hematopoietic progenitors.

As described herein, miR-29a is also over-expressed in human AML. Like human myeloid LSC, miR-29a-expressing myeloid progenitors are capable of serially transplanting the disease. miR-29a can promote progenitor proliferation by expediting G1 to S/G2 phase cell cycle. The results described herein show that miR-29a regulates early events in hematopoiesis. These data also show that miR-29a initiates AML by converting myeloid progenitor into self-renewal LSC.

In one aspect, ectopic expression of miR-29a in mouse HSC/progenitors can be used to cause acquisition of self-renewal capability by myeloid progenitors. In another aspect, ectopic expression of miR-29a in mouse HSC/progenitors can be used to cause biased myeloid differentiation. In still another aspect, ectopic expression of miR-29a in mouse HSC/progenitors can be used to cause the development of a myeloproliferative disorder (MPD) that progresses to acute myeloid leukemia (AML).

miRNA Inhibitors

An “miRNA inhibitory nucleic acid” or an “miRNA inhibitor” is an oligonucleotide that specifically inhibits an miRNA by binding to it. This term includes oligonucleotides composed of naturally occurring nucleobases, sugars, and covalent internucleoside (backbone) linkages as well as oligonucleotides having non-naturally-occurring portions that function similarly. Such modified or substituted oligonucleotides are may be used over native forms because of desirable properties such as, for example, enhanced cellular uptake, enhanced affinity for nucleic acid target, and/or increased stability in the presence of nucleases. The miRNA inhibitory nucleic acids include oligomers or polymers of ribonucleic acid (RNA) or deoxyribonucleic acid (DNA) or both, or modifications thereof. The miRNA inhibitory nucleic acid can be a single-stranded, double stranded, partially double stranded or hairpin oligonucleotide. It preferably consists of, consists essentially of, or comprises at least 12 or more contiguous nucleotides substantially complementary to an endogenous miRNA or a pre-miRNA. As used herein “partially double stranded” refers to double stranded structures that contain fewer nucleotides on one strand. In general, such partial double stranded agents will have less than 75% double stranded structure, less than 50%, or less than 25%, 20% or 15% double stranded structure.

An miRNA inhibitory nucleic acid comprises a region sufficient complementary to the target nucleic acid (e.g., target miRNA, pre-miRNA), and is of sufficient length, such that the miRNA inhibitory nucleic acid forms a duplex with the target nucleic acid. The miRNA inhibitory nucleic acid can modulate the function of the targeted molecule. For example, when the target molecule is an miRNA, such as miR-29a, the miRNA inhibitory nucleic acid can inhibit the gene silencing activity of the target miRNA, which action will up-regulate expression of the mRNA targeted by the target miRNA. When the target is an mRNA, the miRNA inhibitory nucleic acid can replace or supplement the gene silencing activity of an endogenous miRNA.

An miRNA inhibitory nucleic acid can be partially or fully complementary to the target miRNA. It is not necessary that there be perfect complementarity between the miRNA inhibitory nucleic acid and the target, but the correspondence must be sufficient to enable the oligonucleotide agent, or a cleavage product thereof, to modulate (e g, inhibit) target gene expression. The miRNA inhibitory nucleic acid and the target miRNA can have mismatched complementarity at 1, 2, 3, 4, or 5 nucleotide positions.

The miRNA inhibitor can be about 12 to about 33 nucleotides long, preferably, about 15 to about 25, or about 18 to about 25 nucleotides long, or about 21-33 nucleotides long. In certain embodiments, an miRNA inhibitor molecule is 12, 13, 14, 15, 16, 17, 18, 19, 20, 21, 22, 23, 24, 25, 26, 27, 28, 29, 30, 31, 32, or 33 nucleotides in length, or any range derivable therein. Moreover, an miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly an endogenous miRNA. An miRNA-type miRNA inhibitory nucleic acid or pre-miRNA-type miRNA inhibitory nucleic acid can be designed and synthesized to include a region of noncomplementarity (e.g., a region that is 3, 4, 5, or 6 nucleotides long) flanked by regions of sufficient complementarity to form a duplex (e.g., regions that are 7, 8, 9, 10, or 11 nucleotides long) with a target RNA, e.g., an miRNA, such as miR-29a. In a preferred embodiment, the target sequence differs by no more than 1 nucleotide from a sequence shown in any of SEQ ID NO:1-4.

The miRNA inhibitory nucleic acid can be further stabilized against nucleolytic degradation such as by the incorporation of a modification, e.g., a nucleotide modification. The miRNA inhibitory nucleic acid includes a phosphorothioate at least the first, second, or third internucleotide linkage at the 5′ or 3′ end of the nucleotide sequence. In one embodiment, the miRNA inhibitory nucleic acid includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In a particular embodiment, the miRNA inhibitory nucleic acid includes at least one 2′-O-methyl-modified nucleotide, and in some embodiments, all of the nucleotides of the miRNA inhibitory nucleic acid include a 2′-O-methyl modification.

The miRNA inhibitory nucleic acid can be modified so as to be attached to a ligand that is selected to improve stability, distribution or cellular uptake of the agent, e.g., cholesterol. The oligonucleotide miRNA inhibitory nucleic acid can further be in isolated form or can be part of a pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different miRNA.

The miRNA inhibitory nucleic acids featured in the invention can target RNA, e.g., an endogenous pre-miRNA or miRNA of the subject or an endogenous pre-miRNA or miRNA of a pathogen of the subject. For example, the oligonucleotide agents can target an miRNA of the subject, such as miR-29a. Such single-stranded oligonucleotide can be useful for the treatment of diseases involving biological processes that are regulated by miRNAs, including developmental timing, differentiation, apoptosis, cell proliferation, organ development, and metabolism.

An miRNA inhibitory nucleic acid can be constructed using chemical synthesis and/or enzymatic ligation reactions using procedures known in the art. An miRNA inhibitory nucleic acid can be chemically synthesized using naturally occurring nucleotides. Alternatively, it can be synthesized using variously modified nucleotides designed to increase the biological stability of the molecules or to increase the physical stability of the duplex formed between the miRNA inhibitory nucleic acid and target nucleic acids. For example, phosphorothioate derivatives and acridine substituted nucleotides can be used. Other appropriate nucleic acid modifications are described herein. Alternatively, the miRNA inhibitory nucleic acid can be produced biologically using an expression vector into which a nucleic acid has been subcloned in an antisense orientation (i.e., RNA transcribed from the inserted nucleic acid will be of an antisense orientation to a target nucleic acid of interest, such as an miRNA or pre-miRNA).

A preferred miRNA inhibitory nucleic acid is an antagomir, which is a chemically-modified, single-stranded RNA that is antisense to the miRNA sequence. An antagomir is from about 12 to about 33 nucleotides in length, preferably at least about 15 nucleotides in length. A preferred modification is 2′-O-methylation of the ribose. Additional or alternative modifications can include phosphorothioate linkage near 5′ and 3′ ends or a cholesterol moiety conjugated to the 3′ end. Antagomirs form highly stable, sequence-specific duplexes with their corresponding target miRNAs and potently attenuate miRNA activity. In other words, antagomirs act as competitive inhibitors of endogenous target mRNA binding to the miRNA, resulting in suppression of miRNA function. Antagomirs can also induce degradation of target miRNAs. Antagomirs bind to their target miRNAs through sequence-specific base pairing. A morpholino is an example of an antagomir. A locked nucleic acid (LNA) antisense oligonucleotide is also an example of an antagomir. Antagomirs for use in the present invention preferably inhibit miR-29a. In a highly preferred embodiment, the antagomir has the sequence: 5′ ATTTCAGATGGTGCT 3′ (SEQ ID NO: 19).

Antagomirs can be designed according to methods known in the art. See Krützfeldt et al. (2005) and U.S. Publication No. 2009/0092980, incorporated herein by reference. Antagomirs are commercially available, for example, from Ambion, Inc. (Austin, Tex.).

Another preferred miRNA inhibitory nucleic acid is an miRNA sponge (Ebert et al., 2007, incorporated herein by reference). Artificial target RNAs are synthesized to comprise several tandem complementary binding sites to the miRNA to be inhibited. These synthesized RNAs have a bulge or mismatch in the RISC cleavage site and are engineered to be stable in mammalian cells. Like sponges, the artificial RNAs absorb a high number of their complementary miRNAs and release the repression that inhibits translation of corresponding mRNA. The artificial RNAs are prevented from being degraded by miRISCs by the mismatch in the cleavage site. At the same time, the mismatch causes tighter binding between the miRNA and the artificial RNA than between the miRNA and its target mRNA. Sponges can be designed to bind effectively to multiple miRNAs that contain the same seed region. See Tang et al. 2008, incorporated herein by reference, for a review of small RNA technologies, including miRNA inhibition.

### Hematopoietic Cells

The hematopoietic stem cells (HSC) are pluripotent stem cells capable of self-renewal and are characterized by their ability to give rise to cell types of the hematopoietic system. HSC self-renewal refers to the ability of an HSC cell to divide and produce at least one daughter cell with the same self renewal and differentiation potential of a HSC; that is, cell division gives rise to additional HSCs. Self-renewal provides a continual source of undifferentiated stem cells for replenishment of the hematopoietic system.

The marker phenotypes useful for identifying HSCs will be those commonly known in the art. For human HSCs, the cell marker phenotypes include CD34⁺ CD38⁻ CD90(Thy1)⁺Lin⁻. For mouse HSCs, an exemplary cell marker phenotype is Sca-1⁺CD90⁺ (see, e.g., Spangrude, G. J. et al., Science 1:661-673 (1988)) or c-kit⁺Thy^{lo} Lin⁻Sca-1⁺ (see, Uchida, N. et al., J. Clin. Invest. 101(5):961-966 (1998)). Alternative HSC markers such as aldehyde dehydrogenase (see Storms et al., Proc. Nat'l Acad. Sci. 96:9118-23 (1999) and AC133 (see Yin et al., Blood 90:5002-12 (1997) may also be useful.

Differentiation of hematopoietic stem cells can give rise to two different lineages. Lymphoid cells are the cornerstone of the adaptive immune system. They are derived from common lymphoid progenitors. The lymphoid lineage is primarily composed of T-cells and B-cells. (white blood cells). Myeloid cells, which include granulocytes, megakaryocytes, erythrocytes and macrophages, are derived from common myeloid progenitors, and are involved in such diverse roles as innate immunity, adaptive immunity, and blood clotting.

As used herein, “myeloid cell” includes all cells of the myeloid lineage. A myeloid cell can be, for example, a megakaryoblast, a promegakaryocyte, a megakaryocyte, a thrombocyte, a proerythroblast, a basophilic erythroblast, a polychromatic erythroblast, an orthochromatic erythroblast, polychromatic erythrocyte, an erythrocyte, a mast cell, a myeloblast, a promyelocyte, a myelocyte, a metamyelocyte, a basophil, a neutrophil, an eosinophil, a monoblast, a promoonocyte, monocyte, a macrophage, a myeloid dentritic cell, a myeloid leukemia cell and a myeloid leukemia stem cell.

Myeloid cells include common myeloid progenitors. Encompassed within the myeloid progenitor cells are the common myeloid progenitor cells (CMP), a cell population characterized by limited or non-self-renewal capacity but which is capable of cell division to form granulocyte/macrophage progenitor cells (GMP) and megakaryocyte/erythroid progenitor cells (MEP). Non-self renewing cells refers to cells that undergo cell division to produce daughter cells, neither of which have the differentiation potential of the parent cell type, but instead generates differentiated daughter cells. The marker phenotypes useful for identifying CMPs include those commonly known in the art. For CMP cells of murine origin, the cell population is characterized by the marker phenotype c-Kit^{high} (CD 117) CD16^{low} CD34^{low} Sca-1^{neg} Lin^{neg} and further characterized by the marker phenotypes FcyR^{lo} IL-7R.alpha^{neg} (CD127). The murine CMP cell population is also characterized by the absence of expression of markers that include B220, CD4, CD8, CD3, Ter119, Gr-1 and Mac-1. For CMP cells of human origin, the cell population is characterized by CD34⁺CD38⁺ and further characterized by the marker phenotypes CD123⁺ (IL-3R.alpha.) CD45RA^{neg}. The human CMP cell population is also characterized by the absence of cell markers CD3, CD4, CD7, CD8, CD10, CD11b, CD14, CD19, CD20, CD56, and CD234a. Descriptions of marker phenotypes for various myeloid progenitor cells are described in, for example, U.S. Pat. Nos. 6,465,247 and 6,761,883; Akashi, Nature 404: 193-197 (2000); all publications incorporated herein by reference in their entirety.

Further restricted progenitor cells in the myeloid lineage are the granulocyte progenitor, macrophage progenitor, megakaryocyte progenitor, and erythroid progenitor. Granulocyte progenitor cells are characterized by their capability to differentiate into terminally differentiated granulocytes, including eosinophils, basophils, neutrophils. The GPs typically do not differentiate into other cells of the myeloid lineage. With regards to the megakaryocyte progenitor cell (MKP), these cells are characterized by their capability to differentiate into terminally differentiated megarkaryocytes but generally not other cells of the myeloid lineage (see, e.g., WO 2004/024875).

Numerous other suitable cell surface markers are presently known to the skilled artisan, or will be identified and characterized in due course, and such markers will find advantageous use in the methods and compositions described herein. For instance, several additional potential murine markers have recently been identified for the various myeloid progenitor cell populations based on array analysis of mRNA expression. See, e.g., Iwasaki-Arai, et al. J. Exp. Med. 197:1311-1322 (2003); Akashi, et al. Nature 404:193-197 (2000); Miyamoto, et al. Dev. Cell 3:137-147 (2002); Traver, et al. Blood 98:627-635 (2001); Akashi, et al. Blood 101:383-390 (2003); Terskikh, A., et al. Blood 102:102:94-101 (2003). Based on this same type of mRNA expression analysis, additional cell surface markers such as CD 110, CD 114, CD 116, CD 117, CD127, and CD135 may also find use for isolating one or more of the identified myeloid progenitor subpopulations in humans, as described in Manz, et al. Proc Natl Acad Sci USA 99:11872-11877 (2002).

The cells can be derived from any animal species with a hematopoietic system, as generally described herein. Suitable animals can be mammals, including, by way of example and not limitation, rodents (e.g. mice or rats), rabbits, canines, felines, pigs, horses, cows, primates (e.g., human), and the like. The cells for the expansion may be obtained from a single subject, or a plurality of subjects. A plurality refers to at least two (e.g., more than one) donors. When cells obtained are from a plurality of donors, their relationships may be syngeneic, allogenenic, or xenogeneic, as defined herein.

Stem cells and progenitor cells may be mobilized by methods known in the art, for example, from the bone marrow into the peripheral blood by prior administration of cytokines or drugs to the subject (see, e.g., Lapidot, T. et al., Exp. Hematol. 30:973-981 (2002)).

### Ex Vivo Expansion of Myeloid Progenitor Cells

An initial population of cells obtained in a biological sample can be expanded ex vivo in culture by contacting the cells with a medium having a cytokine and growth factor mixture permissive for expansion of myeloid progenitor cells. Cytokines typically act via cellular receptors on the cells modulated by the cytokine Likewise, growth factors in their natural context are also compounds typically made by cells, affecting the proliferation and differentiation of cells, whether the cell is another cell or the cell producing the growth factor. Like cytokines, growth factors generally act on cells via receptors.

For the expansion methods herein, cytokines and growth factors are chosen to expand populations of committed myeloid progenitor cells, such as CMP, GMP, and MEP cells. Since these cells have limited or no self-renewing capacity, the culture conditions are chosen to support division of cells that develop into these myeloid cells while limiting or minimizing growth and expansion of other cell types that are not committed myeloid progenitors.

Accordingly, cytokines for expansion conditions are generally selected from IL-1 (i.e., IL-1.beta.), IL-3, IL-6, IL-11, G-CSF, CM-CSF, and analogs thereof. Forms of the cytokines are naturally occurring products, recombinant products, variants, or modified forms having similar biological activity as the naturally occurring forms such as, e.g., peptide mimetics. The source of the cytokines are chosen to be active on the cells used for expansion, and thus will generally reflect the origin of the initial cells used for expansion. For example, if the progenitor cells are of human origin, human forms of the cytokine, either natural or recombinant, are used. Accordingly, in one embodiment, the cytokines are recombinant human rhulL-1, (i.e., rhulL-1.beta.), rhulL-3, rhulL-6, rhulL-11, rhuG-CSF, rhuGM-CSF, and analogs thereof.

The growth factors for purposes of expansion are selected from stem cell factor (SCF or SF), FLT-3 ligand (FL), thrombopoietin (TPO), erythropoietin (EPO), and analogs thereof. As with the cytokines, growth factor forms are either naturally occurring products or are recombinant forms having similar biological activity as the naturally occurring factors. Accordingly, in one embodiment, the growth factors are recombinant human rhuSCF, rhuFL, rhuTPO, rhuEPO, and analogs thereof.

### Expression Constructs

Nucleic acid molecules can be expressed from transcription units (see for example Couture et al., Trends in Genetics 12:510, 1996) inserted into DNA or RNA vectors. The recombinant vectors can be DNA plasmids or viral vectors. The recombinant vectors capable of expressing the oligonucleotides can be delivered as described herein, and can persist in target cells.

Alternatively, viral vectors can be used that provide for transient expression of nucleic acid molecules. Such vectors can be repeatedly administered as necessary. Viral vectors can be constructed based on, but not limited to, adeno-associated virus, retrovirus, adenovirus, or alphavirus. In another embodiment, Pol II or Pol III-based constructs are used to express nucleic acid molecules of the invention (see for example Thompson, U.S. Pats. Nos. 5,902,880 and 6,146,886). Once expressed, the miRNA inhibitory nucleic acid interacts with the target RNA (e.g., miRNA or pre-miRNA) and inhibits miRNA activity.

### Selectable and Screenable Markers

In certain embodiments of the invention, cells containing a nucleic acid construct of the present invention may be identified in vitro or in vivo by including a marker in the expression vector. Such markers confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP or luciferase, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### Detection Probes and Diagnostic Methods

The miR-29a nucleic acid sequence provided in SEQ ID NO: 1 can be used to create probes to detect the presence of miR-29a in a biological sample. Using such probes, several methods are available for detecting miR-29a expression, such as PCR technology, restriction fragment length analysis, microarray analysis, and oligonucleotide hybridization using, for example, Southern and Northern blotting and in situ hybridization. These processes are well known in the art.

The present invention provides methods of diagnosing a myeloid lineage cell proliferation-related disorder by providing a biological sample from a person suspected of having such a disorder, and determining the level of miR-29a expression in the cells of the biological sample. In particular, this aspect of the invention provides a method of diagnosing such a condition comprising the following steps: (a) contacting a cell sample nucleic acid with a microarray under conditions suitable for hybridization; (b) providing hybridization conditions suitable for hybrid formation between the cell sample nucleic acid and a polynucleotide of the microarray; (c) detecting the hybridization; and (d) diagnosing the disorder condition based on the results of detecting the hybridization. In one non-limiting embodiment, the sample can be selected from the group consisting of blood, an amniocentesis sample, somatically in utero fetal blood, and bone marrow.

Suitable hybridization conditions for the diagnostic methods are those conditions that allow the detection of gene expression from identifiable expression units such as genes. Exemplary stringent hybridization conditions include hybridization at 42° C. in a solution (i.e., a hybridization solution) comprising 50% formamide, 1% SDS, 1 M NaCl, 10% dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press Cold Spring Harbor, N.Y. (1989), pp. 9.47 to 9.51.

Hybridization can be performed at low stringency with buffers, such as 6×SSPE with 0.005% Triton X-100 at 37° C., which permits hybridization between target and probes that contain some mismatches to form target polynucleotide/probe complexes. Subsequent washes can be performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC. Background signals can be reduced by the use of detergent, such as sodium dodecyl sulfate, Sarcosyl or Triton X-100, or a blocking agent, such as salmon sperm DNA.

According to the invention, diagnostic kits can be assembled which are useful to practice oligonucleotide hybridization methods of distinguishing difference in miR-29a expression. Such diagnostic kits comprise a labeled oligonucleotide which hybridizes, for example, to miR-29a. It is contemplated that exemplary probes may be 8, 9, 10, 11, 12, 13, 14, 15, 16, 17, 18, 19, 20, 25, 30, 35, 40, 45, 50, 55, 60, 65, 70, 75, 80, 85, 90, 95, 100 or more contiguous base pairs from the above sequences will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

Labeled probes of the oligonucleotide diagnostic kits according to the invention can be labeled with a radionucleotide. The oligonucleotide hybridization-based diagnostic kits according to the invention can comprise cDNA samples that represent positive and negative controls.

Probes useful as diagnostics can be used not only to diagnose the onset of illness in a patient, but may also be used to assess the status of a patient who may or may not be in remission. It is believed that emergence of a patient from remission is characterized by the presence of cells containing chromosome abnormalities. Thus, patients believed to be in remission may be monitored using the probes of the invention to determine their status regarding progression or remission from disease. Use of such probes will thus provide a highly sensitive assay the results of which may be used by physicians in their overall assessment and management of the patient's illness.

Microarray chips are well known to those of skill in the art (see, e.g., U.S. Pat. Nos. 6,308,170; 6,183,698; 6,306,643; 6,297,018; 6,287,850; 6,291,183, each incorporated herein by reference). DNA-based microarrays provide a simple way to explore the expression of miR-29a in samples from patients in a diagnostic context. Such microarrays also can be used to screen for new miR-29a related sequences. The microarray can be used for large scale genetic or gene expression analysis of a large number of target polynucleotides. The microarray can also be used in the diagnosis of diseases and in the monitoring of treatments. Further, the microarray can be employed to investigate an individual's predisposition to a disease. In addition, the microarray can be employed to investigate cellular responses to infection, drug treatment, and the like.

Microarray expression profiles include a plurality of detectable complexes. Each complex is formed by hybridization of one or more nucleic acids to one or more complementary target polynucleotides. For example, at least one of the miR-29a nucleic acids is hybridized to a complementary target polynucleotide forming at least one of complex. A complex is detected by incorporating at least one labeling moiety in the complex as described above. The expression profiles can show unique expression patterns that are characteristic of the presence or absence of a disease or condition. Such a microarray can be employed in diagnostic, prognostic and treatment applications, drug discovery and development, toxicological and carcinogenicity studies, forensics, pharmacogenomics, and the like.

Microarrays can also be used to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to determine the molecular mode of action of a drug.

### Screening Methods

The present invention also contemplates the screening of candidate miRNA inhibitors, e.g., peptides, polypeptides, nucleic acids or small molecules, for various abilities to mimic, or interfere with the function of the miRNAs described herein. In the screening assays of the present invention, the candidate substance may first be screened for basic biochemical activity (e.g., binding to a target RNA sequence, inhibition of miRNA binding thereto, alteration in gene expression and then further tested for function in at the cellular or whole animal level).

For example, compounds may be screened for their ability to treat a myeloid cell proliferation disorder, or cause, prevent or delay the onset of a myeloid cell proliferation disorder. Accordingly, the present invention provides for a method for determining whether a compound is capable of treating a myeloid lineage cell proliferation related disorder, the method comprising, (a) administering a test compound to a non-human mammal; (b) measuring expression of miR-29a in a myeloid lineage cell of the non-human mammal of (a), and (c) comparing the expression of miR-29a measured in (b) to the expression of miR-29a measured in myeloid lineage cell of a sibling of the non-human mammal, wherein the sibling was not administered the test compound, and wherein a decrease in the expression of miR-29a in the non-human mammal of (a) compared to (b) indicates the that the test compound is capable of treating a myeloid lineage cell proliferation related disorder. In one embodiment, the non-human mammal has a myeloid cell proliferation disorder. In another embodiment, the non-human mammal has AML. In another embodiment the non-human mammal has CML.

In screening for related RNA molecules with inhibitory activity, the hybridization conditions will generally be selected to mimic those in in-cyto environments. By way of reference, “stringent conditions” are those that allow hybridization between two homologous nucleic acid sequences, but preclude hybridization of random sequences. Hybridization at high temperature and/or low ionic strength is termed high stringency. In contrast, hybridization at low temperature and/or high ionic strength is termed “low stringency,” which permits hybridization of less related sequences. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. Other factors that can affect stringency are the presence of formamide, tetramethylammonium chloride and/or other solvents in the hybridization mixture.

### Treatment Methods and Routes of Delivery

A nucleic acid may be delivered to an organelle, a cell, a tissue or an organism via one or more injections (i.e., a needle injection), such as, for example, subcutaneously, intradermally, intramuscularly, intravenously, intraperitoneally, etc. The nucleic acid can also be introduced into a cell by direct microinjection. (Harland and Weintraub, 1985). The amount of nucleic acid used may vary upon the nature of the antigen as well as the organelle, cell, tissue or organism used.

The nucleic acid can also be introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

In the present methods, the nucleic acid can be administered either as a naked oligonucleotide agent, in conjunction with a delivery reagent, or as a recombinant plasmid or viral vector which expresses the oligonucleotide agent.

The nucleic acid can include a delivery vehicle, such as liposomes, hydrogels, cyclodextrins (see for example Gonzalez et al., Bioconjugate Chem. 10: 1068, 1999), biodegradable nanocapsules, and bioadhesive microspheres, or by proteinaceous vectors (O'Hare and Normand, International PCT Publication No. WO 00/53722) for administration to a subject, carriers and diluents and their salts, and/or can be present in pharmaceutically acceptable formulations. Contemplated in the present invention are various commercial approaches involving “lipofection” technology. In certain embodiments of the invention, the liposome may be complexed with a hemagglutinating virus (HVJ). This has been shown to facilitate fusion with the cell membrane and to promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991).

Methods for the delivery of nucleic acid molecules are described in Akhtar et al., Trends in Cell Bio. 2:139, 1992; Delivery Strategies for Antisense Oligonucleotide Therapeutics, ed. Akhtar, 1995; Maurer et al., Mol. Membr. Biol., 16:129, 1999; Hofland and Huang, Handb. Exp. Pharmacol. 137:165, 1999; and Lee et al., ACS Symp. Ser. 752:184, 2000. Beigelman et al., U.S. Pat. No. 6,395,713 and Sullivan et al., PCT WO 94/02595 further describe the general methods for delivery of nucleic acid molecules.

Other methods known to those of skill in the art include ionophoresis, direct delivery of DNA, such as by ex vivo transfection (Wilson et al., 1989, Nabel et al, 1989), injection (U.S. Pat. Nos. 5,994,624, 5,981,274, 5,945,100, 5,780,448, 5,736,524, 5,702,932, 5,656,610, 5,589,466 and 5,580,859), including microinjection (Harland and Weintraub, 1985; U.S. Pat. No. 5,789,215); electroporation (U.S. Pat. No. 5,384,253; Tur-Kaspa et al., 1986; Potter et al., 1984); calcium phosphate precipitation (Graham and Van Der Eb, 1973; Chen and Okayama, 1987; Rippe et al., 1990); DEAE-Dextran followed by polyethylene glycol (Gopal, 1985); direct sonic loading (Fechheimer et al, 1987); microprojectile bombardment (PCT Application Nos. WO 94/09699 and 95/06128; U.S. Pat. Nos. 5,610,042; 5,322,783 5,563,055, 5,550,318, 5,538,877 and 5,538,880); agitation with silicon carbide fibers (Kaeppler et al., 1990; U.S. Pat. Nos. 5,302,523 and 5,464,765); desiccation/inhibition-mediated DNA uptake (Potrykus et al., 1985); and any combination of such methods.

In certain embodiments of the invention, the nucleic acid can be expressed from an expression construct comprised in a virus or an engineered construct derived from a viral genome. The ability of certain viruses to enter cells via receptor-mediated endocytosis, to integrate into host cell genomes and express viral genes stably and efficiently have made them attractive candidates for the transfer of foreign genes into mammalian cells (Ridgeway, In: Rodriguez R L, Denhardt D T, eds. Vectors: A survey of molecular cloning vectors and their uses. Stoneham: Butterworth, pp. 467 492, 1988; Nicolas et al., In: Vectors: A survey of molecular cloning vectors and their uses, Rodriguez & Denhardt (eds.), Stoneham: Butterworth, pp. 493 513, 1988; Baichwal et al., In: Gene Transfer, Kucherlapati ed., New York, Plenum Press, pp. 117-148, 1986; Temin, In: gene Transfer, Kucherlapati (ed.), New York: Plenum Press, pp. 149 188, 1986). The viral vector can be derived from any suitable virus known in the art. Such vectors include retroviral vectors, such as lentiviral, adenoviral, baculoviral, avian, and mouse stem cell viral viral vectors, adenoviral vectors, poxviral vectors, and vaccinia viral vectors.

Other vector delivery systems which can be employed to deliver a nucleic acid encoding a given gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu et al., 1993).

The miRNA inhibitory nucleic acid (e.g., miR-29a) can be delivered to a subject by any route known in the art. Administration can be local or systemic. It can be topical (including ophthalmic, intranasal, transdermal, intrapulmonary), oral or parenteral. Parenteral administration includes intravenous drip, subcutaneous, intraperitoneal or intramuscular injection, or intrathecal or intraventricular administration.

Formulations for direct injection and parenteral administration are well known in the art. Such formulations may include sterile aqueous solutions which may also contain buffers, diluents and other suitable additives. For intravenous use, the total concentration of solutes should be controlled to render the preparation isotonic. The miRNA inhibitor can be administered orally or by intramuscular injection or by intravenous injection, in admixture with a pharmaceutically acceptable carrier adapted for the route of administration.

The nucleic acid can be administered in a single dose or in multiple doses. Where the administration of the nucleic acid is by infusion, the infusion can be a single sustained dose or can be delivered by multiple infusions. Injection of the nucleic acid can be directly into the tissue at or near the site of aberrant or unwanted target gene expression (e.g., aberrant or unwanted miRNA or pre-miRNA expression). Multiple injections of the agent can be made into the tissue at or near the site.

One skilled in the art can determine an effective amount of the miRNA inhibitory nucleic acid specific to miR-29a of the invention to be administered to a given subject, by taking into account factors such as the size and weight of the subject; the extent of the cancer progression or disease penetration; the age, health and sex of the subject; the route of administration; and whether the administration is regional or systemic.

In addition to treating pre-existing diseases or disorders, oligonucleotide agents featured in the invention (e.g., single-stranded oligonucleotide agents targeting miR-29a) can be administered prophylactically in order to prevent or slow the onset of a particular disease or disorder. In prophylactic applications, an miRNA inhibitory nucleic acid is administered to a patient susceptible to or otherwise at risk of a particular disorder, such as disorder associated with aberrant or unwanted expression of an miRNA or pre-miRNA.

Ex vivo methods, which involve isolation of a cell from a subject, manipulation of the cell outside of the body, and reimplantation of the manipulated cell into the subject can also be used in conjunction with the methods described herein. The ex vivo procedure may be used on autologous or heterologous cells. In some embodiments, the ex vivo method is performed on cells that are isolated from bodily fluids such as peripheral blood or bone marrow, but may be isolated from any source of cells. When returned to the subject, the manipulated cell will be programmed for cell death or division, depending on the treatment to which it was exposed. Ex vivo manipulation of cells has been described in several references in the art, including Engleman, E. G., 1997, Cytotechnology, 25:1; Van Schooten, W., et al., 1997, Molecular Medicine Today, June, 255; Steinman, R. M., 1996, Experimental Hematology, 24, 849; and Gluckman, J. C., 1997, Cytokines, Cellular and Molecular Therapy, 3:187. The ex vivo activation of cells of the invention may be performed by routine ex vivo manipulation steps known in the art. In vivo methods are also well known in the art. The invention thus is useful for therapeutic purposes and also is useful for research purposes such as testing in animal or in vitro models of medical, physiological or metabolic pathways or conditions.

The following examples are provided to illustrate aspects of the invention and are non-limiting.

### EXAMPLES

### Example 1

### Methods

### Human Bone Marrow and Peripheral Blood Samples

Normal bone marrow samples from healthy donors <35 years old were purchased from All Cells, Inc (Berkley, Calif.). Bone marrow mononuclear cells were prepared by Ficoll density centrifugation and enriched for CD34⁺ cells by magnetic bead selection prior to staining according to the manufacturer's protocol (CD34⁺ Microbead Kit, Miltenyi Biotec). Primary human AML samples were collected from the Stanford Hospital Clinical Laboratory under an IRB approved protocol (#11177).

### Retroviral Preparation and Transduction

miR-29a was PCR-amplified from mouse genomic DNA using the following primers: 1) 5′CCGCTCGAGTTGGTTTGGCCCTTTATC (SEQ ID NO: 5), 2) 5′CGGAATTCCCACCCTGCTTACCTCTG (SEQ ID NO: 6). The resulting fragment was cloned into the XhoI and EcoRI sites in the 3′LTR of MDH-PGK-GFP, and the insert was confirmed by sequencing. Retroviral supernatant was generated by standard procedures following calcium phosphate transfection of MDH-PGK-GFP2.0-miR-29a and the pCLeco viral packaging construct into 293T cells.

To enrich for hematopoietic stem/progenitor cells, donor mice were injected i.p. with 5 mg 5-fluorouracil 5 days before bone marrow harvest. BM cells were collected by flushing long bones with PBS/1% FBS and red blood cells were lysed with ACK lysis buffer (Loundon). BM cells were infected with retrovirus per previously described protocol (Chen et al., 2004). Infected cells were resuspended in PBS then injected i.v. into lethally irradiated (9.5 Gy) recipient mice.

miRNA Analysis

Total RNA was prepared from sorted human populations using miRvana RNA prep kits (Ambion) according to the manufacturer's protocol. 100 ug total RNA was pre-amplified 14-18 rounds using a set of 315 unique probes designed by Applied Biosystems (Chen et al., 2004), and the resulting reaction product was divided equally into a 384-well plate prespotted with individual miRNA TaqMan probes. PCR was performed for 40 cycles, 1′ at 95° C., 30 sec at 60° C.

Mature miR-29a expression was measured using the mirVana qRT-PCR miRNA Detection kit (Ambion). In brief, total RNA was isolated using Trizol (Invitrogen). cDNA was synthesized using U6 or miR-29a specific reverse transcription primer sets, followed by application with their respective PCR primer sets using SYBR Green (Roche). ROX Reference dye was used for normalization of fluorescent reporter signal (Invitrogen). Expression levels were normalized to endogenous expression of U6 snRNA.

Northern blots were prepared using 20 ug of total RNA. A γ-32P-ATP end-labeled DNA anti-sense oligonucleotide probe to miR-29a was used for detection. Standard hybridization conditions were utilized and washes were performed at room temperature. U6 expression was used as a loading control.

### Mice/Transplantations

C57B1/6 and C57B1/6 SJL mice were purchased from Taconic or kindly provided by YR. Zou, respectively. B6/Ka and B6 Ly5.2 mice were bred in I.L.W.'s mouse colony. Cells for transplant were injected intravenously into the retro-orbital sinuses of recipient mice under isofluorane or avertin anesthesia. Recipient mice were sublethally irradiated (4.7 Gy) or lethally irradiated (9.5 Gy) using a cesium radiation source and were maintained on antibiotics (Baytril, Bayer) at least 4 weeks post-transplantation. All mice used in this study were maintained under specific pathogen-free conditions according to institutional guidelines and animal study proposals approved by the institutional animal care and use committees.

### Mouse Tissues

Bone marrow cells were prepared by crushing long bones and pelvis with a mortar and pestle in staining media (PBS/2% fetal calf serum). Splenocyte cell suspensions were prepared by mechanical dissociation. Red cell lysis was performed with ACK lysis buffer prior to flow cytometric analysis. For histology, samples were fixed in 4% paraformaldehyde or 10% neutral-buffered formalin overnight prior to standard processing for paraffin-embedded tissues. 10 um sections were stained with hematoxylin-eosin. Cytospin preparations were prepared by spinning cells in a Shandon cytocentrifuge (Thermo) at 500 rpm for 5′. Slides were stained with a modified Wright-Giemsa stain per standard protocols.

### Cell Staining and Flow Cytometry

Human bone marrow cells were stained for HSC/progenitor populations as previously described (Majeti et al., 2007b) (Manz et al., 2002). Following staining, cells were analyzed and sorted using a FACSAria (Becton Dickinson), with purity routinely >90%. Primary human AML LSC (Lin⁻CD34⁺CD38⁻) and non-LSC (Lin⁻CD34⁺CD38⁺) blasts were stained using a more limited lineage antibody cocktail (CD3, CD4, CD8, CD19, CD20, CD14, CD11b).

Mouse stem and progenitor cell stains included the following monoclonal antibodies: lineage cocktail—Mac-1, Gr-1, CD3, CD4, CD8, B220, and Ter119 conjugated to Cy5-PE (eBioscience); c-Kit PE-Cy7 or APC-Cy7 (eBioscience); Sca-1 Alexa680 or Pacific Blue (e13-161-7); CD34 FITC or biotin (eBioscience); CD16/32 (FcγRI/III) APC (Pharmingen); and CD135 (Flk-2) PE (eBioscience). Staining was performed as previously described (Akashi et al., 2000) (Yang et al., 2005). Cells were then stained with streptavidin conjugated PECy7 (eBioscience) or quantum dot 605 (Chemicon). FACS data was analyzed using FlowJo software (Treestar).

### Cell Cycle Assays

For BrdU incorporation assays, 10 uM BrdU was added to equal numbers of wild type 293T cells and 293T cells stably expressing miR-29a cells (in duplicate for each time point). Cells were collected at indicated time points, and processed according to the manufacturer's protocol using APC-anti-BrdU and 7-AAD (BD Bioscience).

### Microarray Analysis and Validation

Spleen myeloid cells (Mac-1⁺Gr-1⁺) from empty vector control or mir-29a chimeric mice were sorted using a FACSAria. Total RNA was extracted using RNeasy total RNA kit (Qiagen) and cRNA was produced from the RNA for array analysis using the Illumina TotalPrep RNA Amplification kit according to the manufacturer's protocol (Ambion). The labeled cRNA was hybridized using the Illumina array at Rockefeller University, Genomics Resource Center.

For semiquantitative RT-PCR, cDNA was made using random primers and the Superscript reverse transcription kit (Invitrogen) according to the manufacturer's protocol. The following PCR primers were used for detection. β-actin was used for normalization.

| (SEQ ID NO: 7) | |
|---|---|
| Hdac6 | 5′-CATCAGAGAGCAACTGATCC, |
| | 5′-ATGCCATTCCGAATCTCAGC. |
| (SEQ ID NO: 8) | |
| Bak1 | 5′-GCTACGTTTTTTACCTCCAC, |
| | 5′-CATCTGGCGATGTAATGATG. |
| (SEQ ID NO: 9) | |
| Lrrk1 | 5′-GTGAGTCTTTGGAGGTCCTT, |
| | 5′-TGATGGTGGCTTCACTGTGT. |
| (SEQ ID NO: 10) | |
| Slfn4 | 5′-TCCACAGACATCCATAGAGC, |
| | 5′-TTCCATCTCTGGTGAAGCTG. |
| (SEQ ID NO: 11) | |
| Cdc42ep2 | 5′-TCTGCTCAAAAACGCCATCT, |
| | 5′-GTGATCCATAATCTGGAGGA . |
| (SEQ ID NO: 12) | |
| Hbp1 | 5′-CACCATTTGGCACTGCTTTC |
| | 5′-CCGAATGACACACTCTCTTC. |
| (SEQ ID NO: 13) | |
| Actin | 5′-GCTACAGCTTCACCACCACAG |
| | 5′-GGTCTTTACGGATGTCAACGTC. |

Western blots were performed per standard protocols and protein was revealed by chemiluminescence per manufacturer's protocol (ECL, Amersham). Antibodies included the Hbp1 (1:800, Santa Cruz Biotechnology)

pGL3 firefly luciferase reporter constructs were generated by cloning the 3′UTR of respective genes downstream of the luciferase ORF. Constructs (0.05 ug each) were co-transfected into 293T with a Renilla luciferase control vector (0.01 ug) by calcium phosphate transfection. Luciferase activity was measured 36 h post transfection and normalized against Renilla activity according to manufacturer's protocol (Dual-Luciferase Reporter Assay System, Promega). Constructs were generated using the following primers:

| (SEQ ID NO: 14) | |
|---|---|
| Hbp1 3UTR | 5′ CTAGTCTAGATGCTTGTGTTTGTAAGTCTG |
| | 5′-CTAGTCTAGAGGGGCAATATGTTTAACAAG. |
| (SEQ ID NO: 15) | |
| Bak1 3UTR | 5′-GCTCTAGAGCATTGCACAGTTTATTTCCA |
| | 5′-GCTCTAGACTGGCTGGACTAAACCTCT. |
| (SEQ ID NO: 16) | |
| Dpp3 3UTR | 5′-GGACTAGTGAAGATCTGTGTGGTCTCTC |
| | 5′-GGACTAGTGATGGTGGTCGTCATATTTATT. |
| (SEQ ID NO: 17) | |
| E2F7 3UTR | 5′-GCTCTAGATGCTTCGGTGGGTGGGAT |
| | 5′-GCTCTAGATAACATACACGTCTTACTAAATA. |
| (SEQ ID NO: 18) | |
| Ptpn7 3UTR | 5′-GGACTAGTCCCTCCACCAGCTCATGG |
| | 5′-GGACTAGTGCATCCAAGATGGTTATTTATTTA. |

### Example 2

### Differential Expression of miR-29a in Long-Term Hematopoietic Stem Cells and Committed Hematopoietic Progenitors

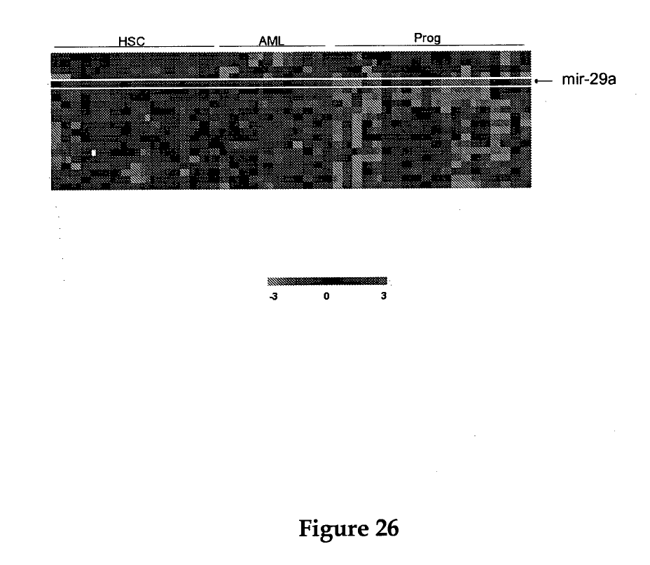

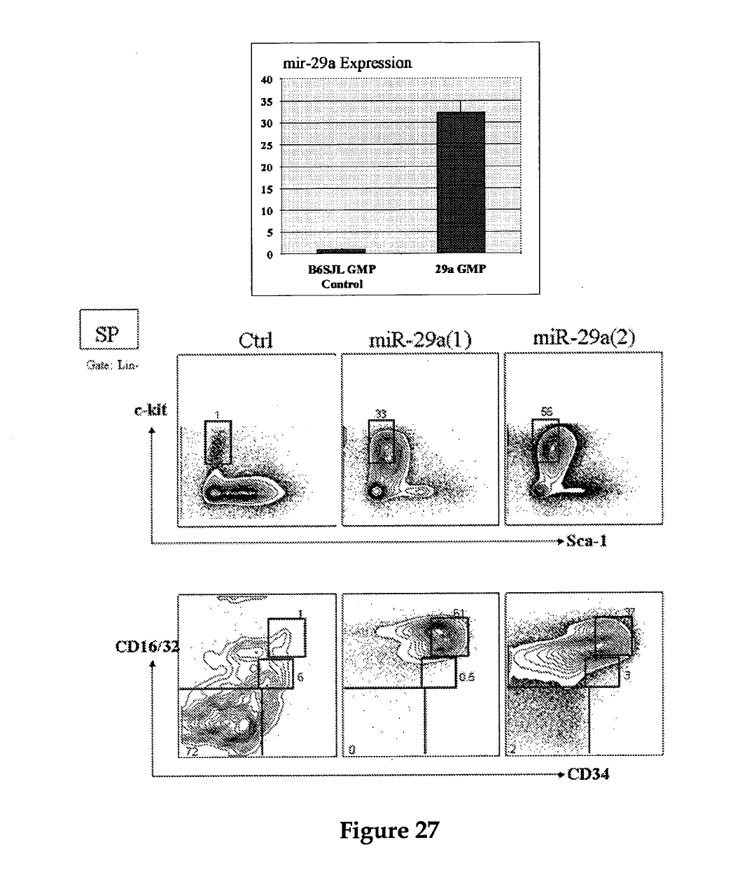

In order to evaluate the function of miRNAs in early hematopoietic development, miRNA expression levels were measured in various lineages of mouse hematopoietic cells were analyzed by microarray and the impact of altered miRNA expression on lineage specification and transformation was assessed by genetic approaches. MicroRNA expression of 315 miRNAs was measured in highly purified populations of normal HSC, MPP, and lineage-committed progenitors, including CLP, CMP, granulocyte-macrophage progenitors (GMP), and megakaryocytic-erythrocyte progenitors (MEP), as well as in leukemia stem cells (LSC) and non-LSC blasts. Mature miRNA expression was measured using a highly sensitive multiplexed TaqMan based real time PCR method (Chen et al., 2005) (FIG. 26). Expression was normalized against an endogenous small RNA (sno-R2) expressed at similar levels in the tested cell populations (Table 1 and Table 2).

TABLE 1
Choice of snoR-02 as an endogenous control for normalization
of miRNA expression levels. Summary of correlation coefficients
from TaqMan based RT-PCR analysis of six candidate small
RNAs endogenous controls in normal human HSC/progenitors
and AML LSC and non-LSC blasts.

| | hsa- | hsa- | hsa- | hsa- | snoR- | sno- |
|---|---|---|---|---|---|---|
| | let-7f | miR-19b | miR-28 | miR-30d | 02 | R-13 |
| has-let-7f | 1 | | | | | |
| has-miR-19b | 0.91 | 1 | | | | |
| has-miR-28 | 0.93 | 0.91 | 1 | | | |
| has-miR-30d | 0.9 | 0.95 | 0.91 | 1 | | |
| snoR-02 | 0.92 | 0.92 | 0.91 | 0.93 | 1 | |
| sno-R-13 | 0.79 | 0.76 | 0.83 | 0.74 | 0.84 | 1 |

TABLE 2
Summary of candidate small RNA expression variation among normal
human HSC/progenitors and AML LSC and non-LSC blasts.

| | hsa- | hsa- | hsa- | hsa- | snoR- | sno- |
|---|---|---|---|---|---|---|
| | let-7f | miR-19b | miR-28 | miR-30d | 02 | R-13 |
| Median Ct | 24 | 16.2 | 24.7 | 21 | 19 | 11.3 |
| stdev_all | 6.4 | 3 | 5.5 | 3.7 | 3 | 2.2 |
| stdev_AML | 1.4 | 1.5 | 1.7 | 1.8 | 1.3 | 0.7 |
| stdev_normal | 5.7 | 2.7 | 5.1 | 2.6 | 2.3 | 2.2 |

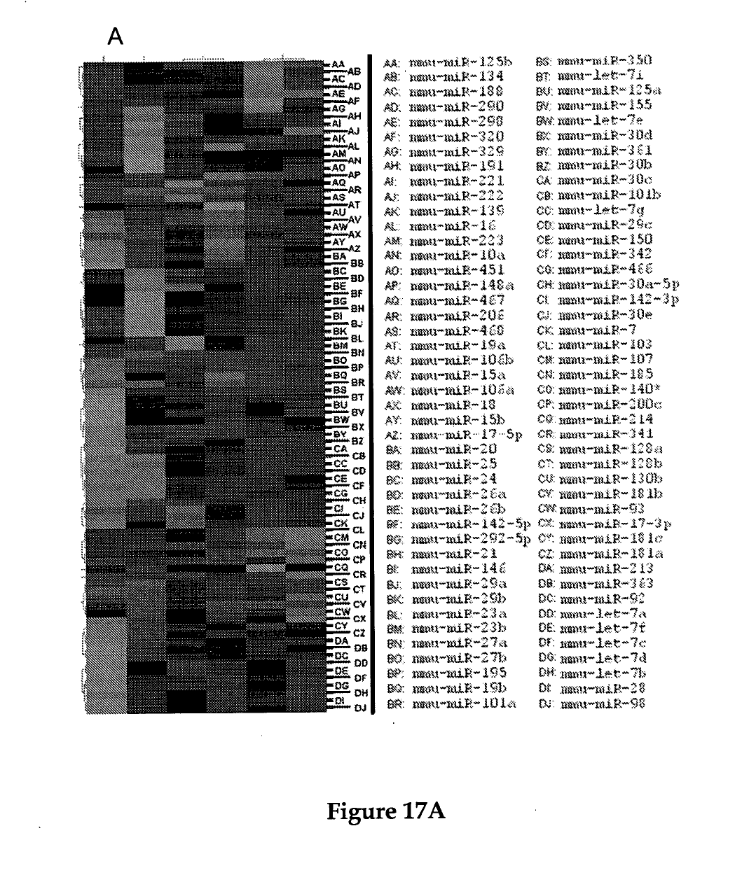

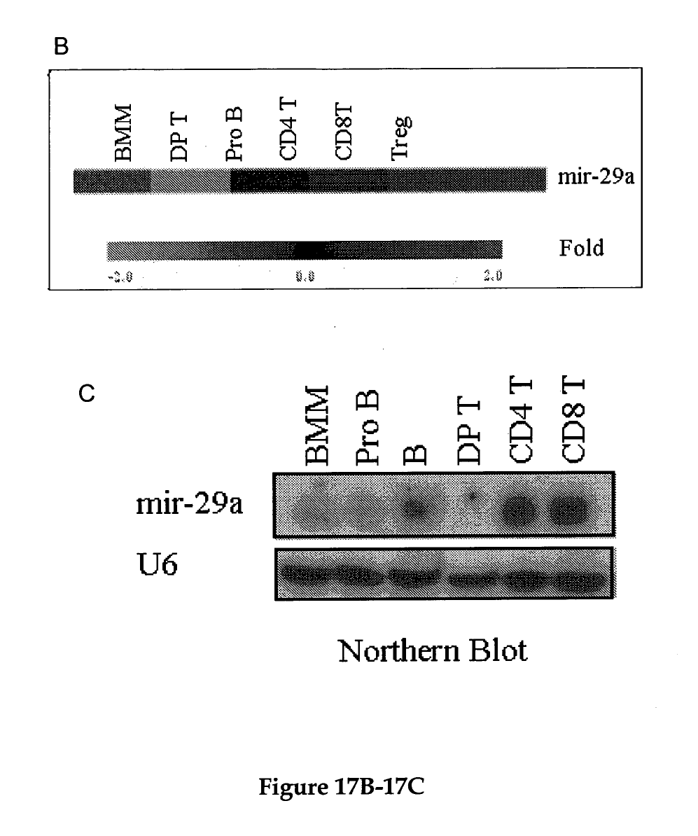

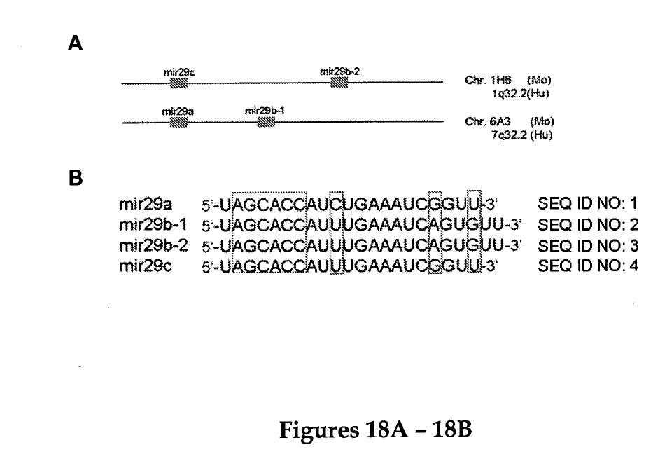

Multiple miRNAs were differentially expressed in myeloid and lymphoid lineages. Amongst them, miR-29a was moderately expressed in bone marrow (BM) myeloid cells but highly expressed in various lineages of mature lymphocytes. miR-29a was expressed at high levels in human AML as well as normal human HSC (FIG. 17).

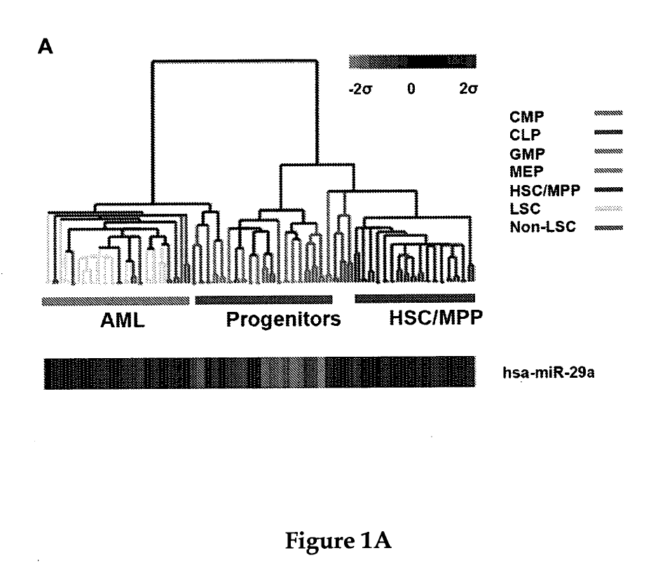

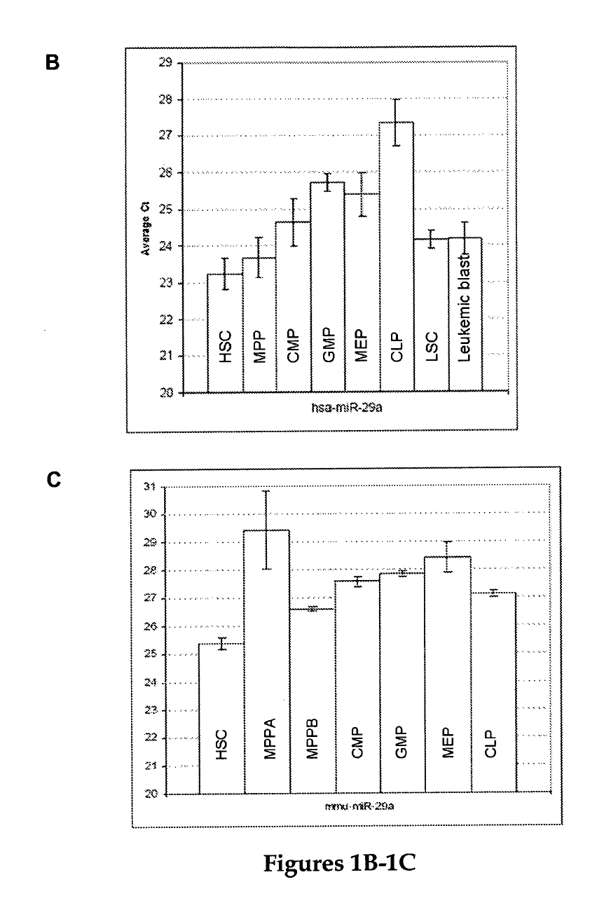

Using unsupervised clustering and SAM analysis with a stringent cutoff (FDR <1%), miR-29a consistently showed higher expression levels in HSC and MPP compared to more committed myeloid and lymphoid progenitors, with HSC/MPP showing about 4× higher expression than the progenitor populations (FIGS. 1A, 1B). This pattern of expression was similar to that seen in mouse HSC and committed progenitors (FIG. 1C). These data show that miR-29a exhibits an evolutionarily conserved pattern of expression with the highest expression found in the most primitive hematopoietic cells and indicates that the level of miR-29a expression can contribute to HSC function.

### Example 3

### Over-expression of miR-29a in Hematopoietic System Leads to Biased Myelopoiesis and Myeloid proliferative disorders

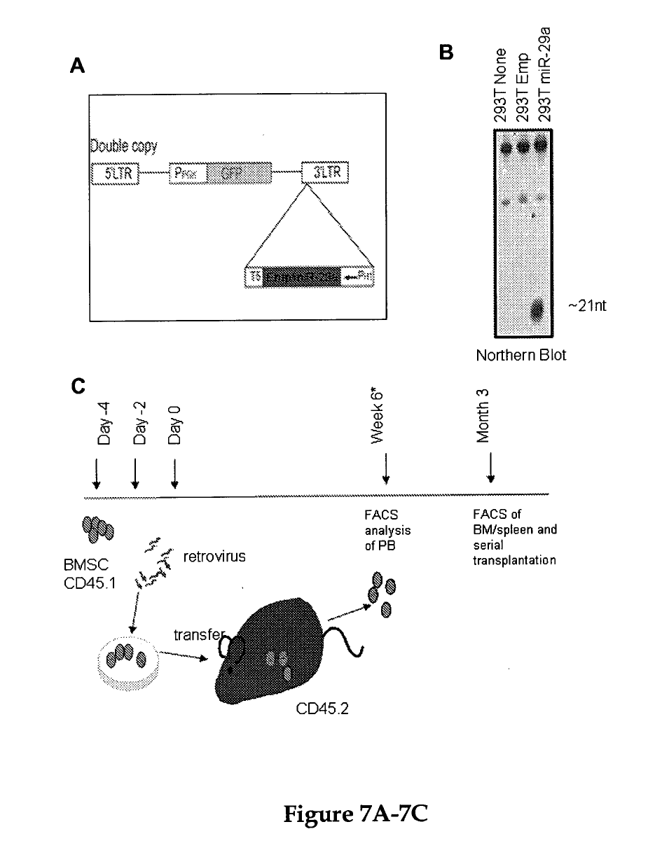

In order to assess miR-29a function in the hematopoietic system, mouse bone marrow cells enriched for HSC/progenitor cells were transduced with mouse miR-29a or empty control MSCV-based, GFP-expressing retroviruses. The transduction efficiency ranged between 30-80% of HSC enriched population. Transduced cells were transplanted into lethally irradiated recipients. Expression and appropriate processing of miR-29a were confirmed by RT-PCR and Northern blot analysis (FIG. 7A-7C).

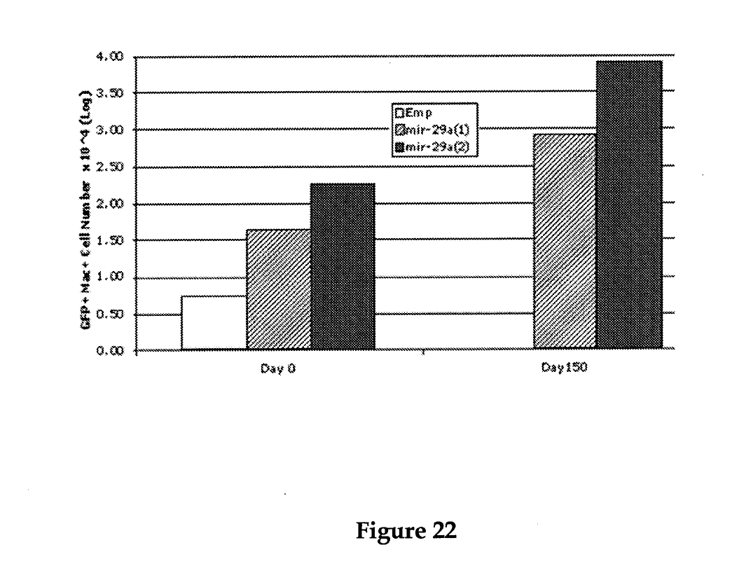

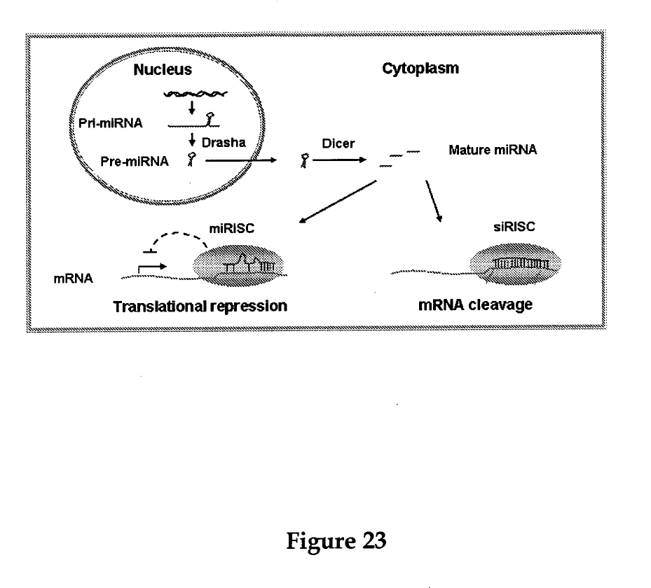

Over-expression of miR-29a in hematopoietic cells resulted in a marked expansion of myeloid (Mac-1⁺Gr-1⁺) cells in both the BM and periphery (FIG. 22). In contrast, development of B cells was blocked whereas T cell development was not significantly altered in the same chimeras.

TABLE 7
High Penetrance of Myeloid Leukemia by miR-29a Overexpression.
Incidence of myeloid leukemia in miR-29a BM chimera mice. Shown
are summary of myeloid leukemia incidence in miR-29a overexpressing
BM chimeras from three independent experiments. While empty vector
infected BM chimeras showed no signs of myeloid leukemia, 82% of
miR-29a overexpressing BM chimera mice developed myeloid leukemia
like phenotypes. Myeloid leukemia like phenotype based on white blood
cell counting and histological analysis.

| | Exp | | | |
|---|---|---|---|---|
| | 1 | 2 | 3 | |
| Vector | 0/6 | 0/4 | 0/4 | |
| 29a | 7/8 | 3/4 | 4/5 | 82% |

The peripheral expanded Mac1⁺ Gr1⁺ cells contained a significant proportion (about 10% to about 30%) of immature monocytes. These cells were actively dividing and expanded markedly in recipient mice even after three series of adoptive transfer. Serial transplantablility confirmed the presence of a leukemia stem cell population resulting from over-expression of miR-29a. Histopathological analysis indicated that these mice developed symptom resemblance of human chronic myeloid leukemia (CML).

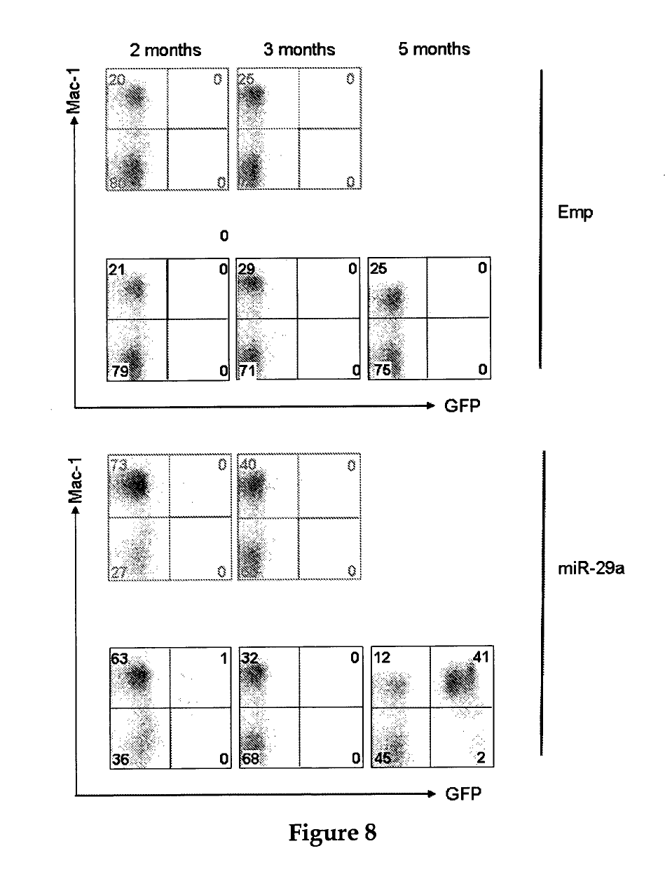

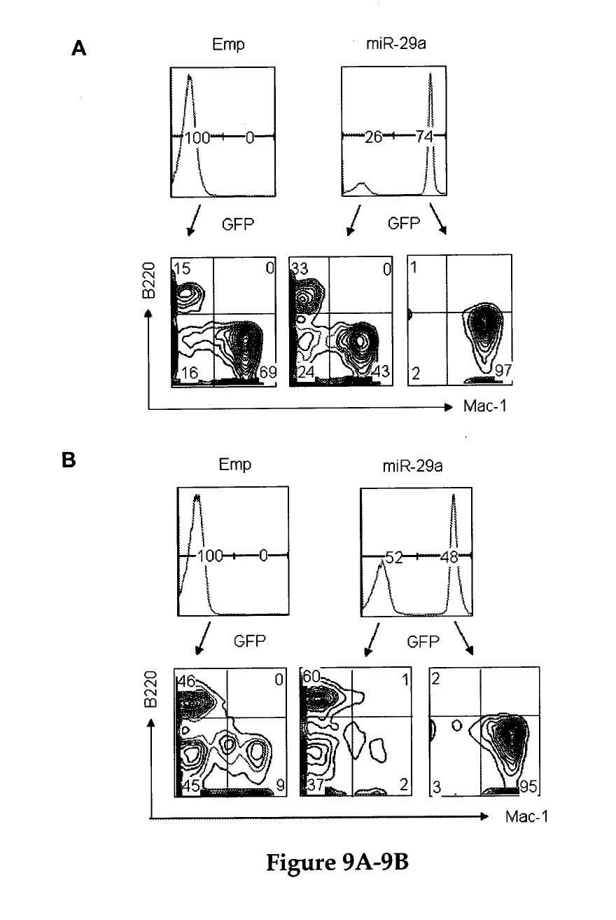

Flow cytometric analysis of the peripheral blood in stably engrafted primary chimeric mice showed increasing levels of donor-derived myeloid (Mac-1⁺) chimerism 8-12 weeks post-transplantation, as well as reduced percentages of donor-derived B cells (FIGS. 8 and 9). This effect was specific to miR-29a, as neither empty retrovirus nor retroviruses expressing miR-24-2 or miR-27a significantly affected engraftment levels or lineage output when transduced into mouse HSC/progenitors.

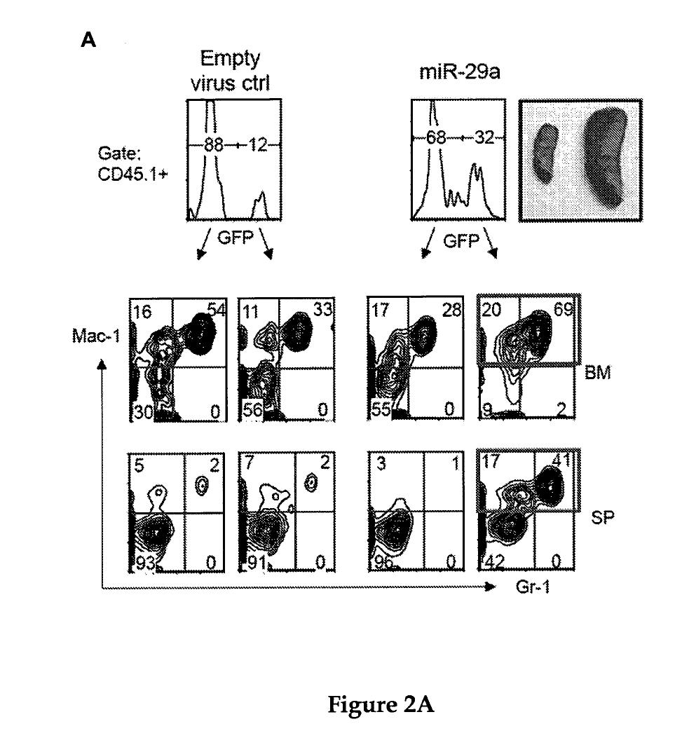

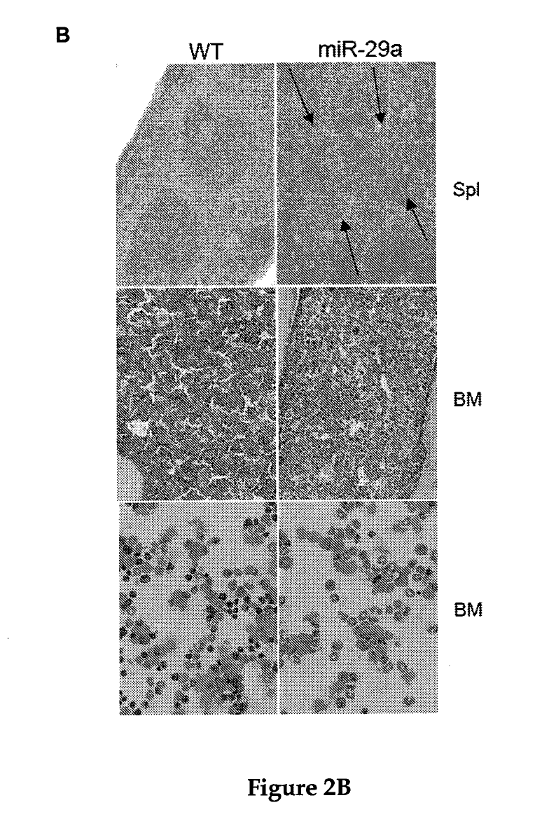

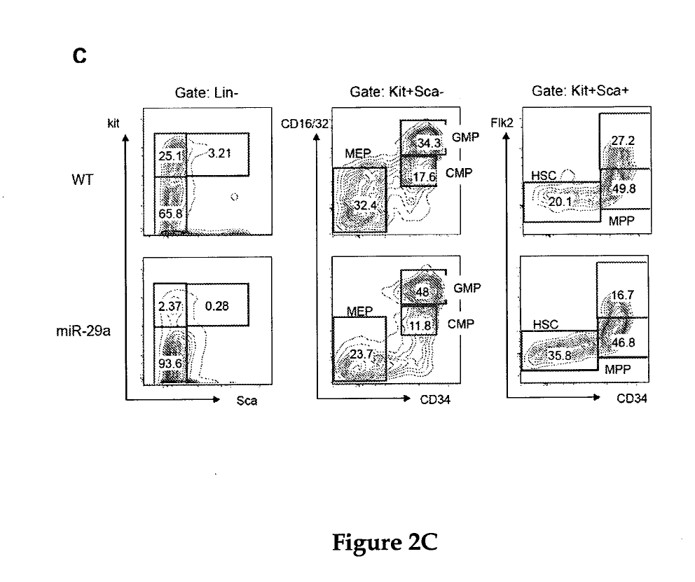

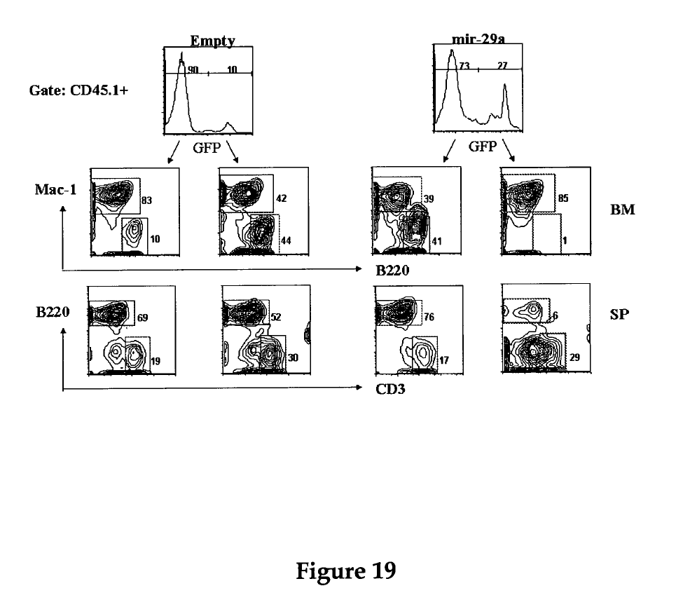

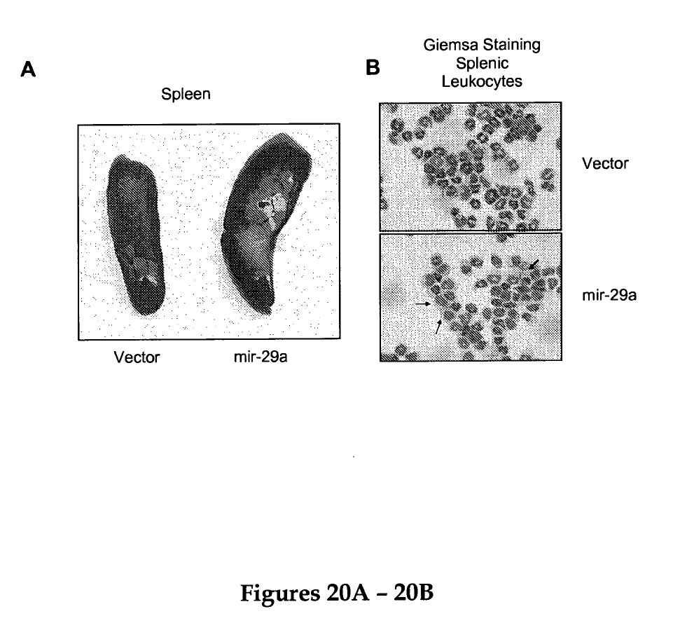

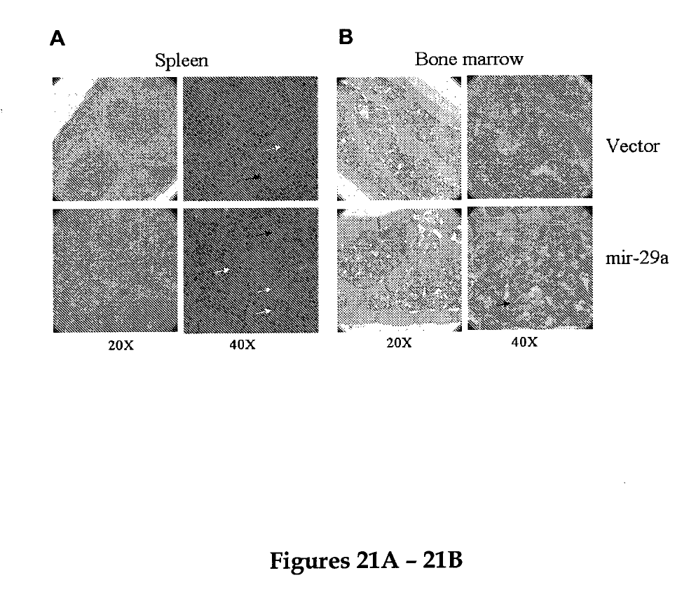

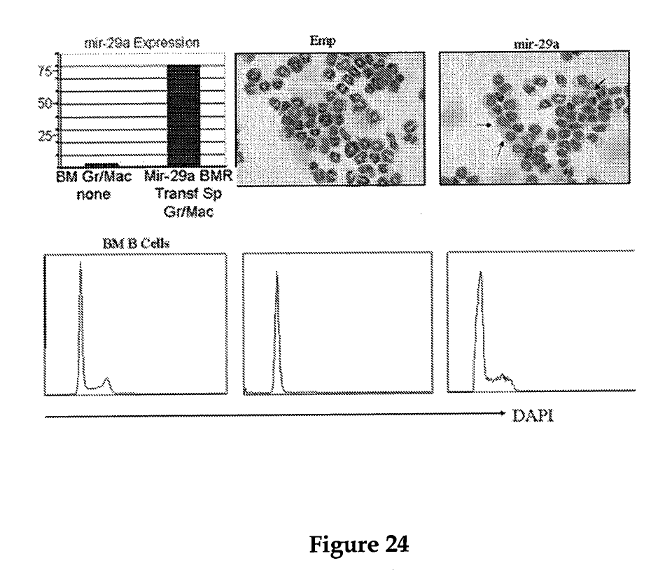

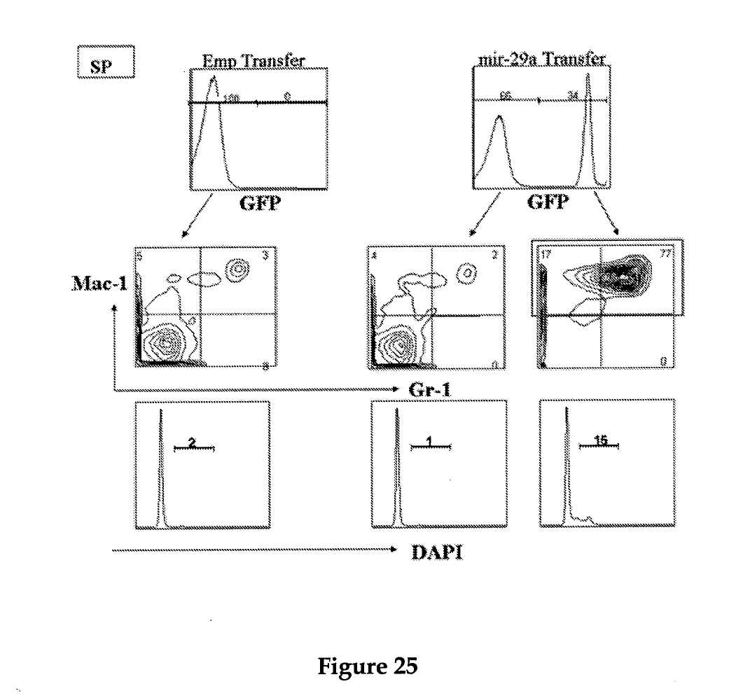

Stably engrafted primary recipients (>16 weeks post-transplant) were necropsied to evaluate engraftment. Chimeric mice exhibited mild splenomegaly, and flow cytometric analysis of splenocytes and bone marrow cells revealed a marked myeloid lineage bias with expansion of donor-derived granulocytes (Mac-1⁺Gr-1⁺) and monocytes (Mac-1⁺Gr-1^{lo-int}) as well as decreased production of B lymphocytes (FIG. 2A; FIG. 9; FIG. 19; FIG. 20; FIG. 24). Histologic sections of the spleen showed expanded red pulp and increased extramedullary hematopoiesis with prominent granulocytic and megakaryocytic hyperplasia (FIG. 21A). Sections and cytospin preparations of the bone marrow showed granulocytic and megakaryocytic hyperplasia with a significant decrease in erythroid precursors (FIG. 2B; FIG. 21B). Taken together, these findings were consistent with a MPD.

### Example 4

### miR-29a-Induced Biased Myelopoiesis is Established at Myeloid Progenitor Stages During Hematopoiesis

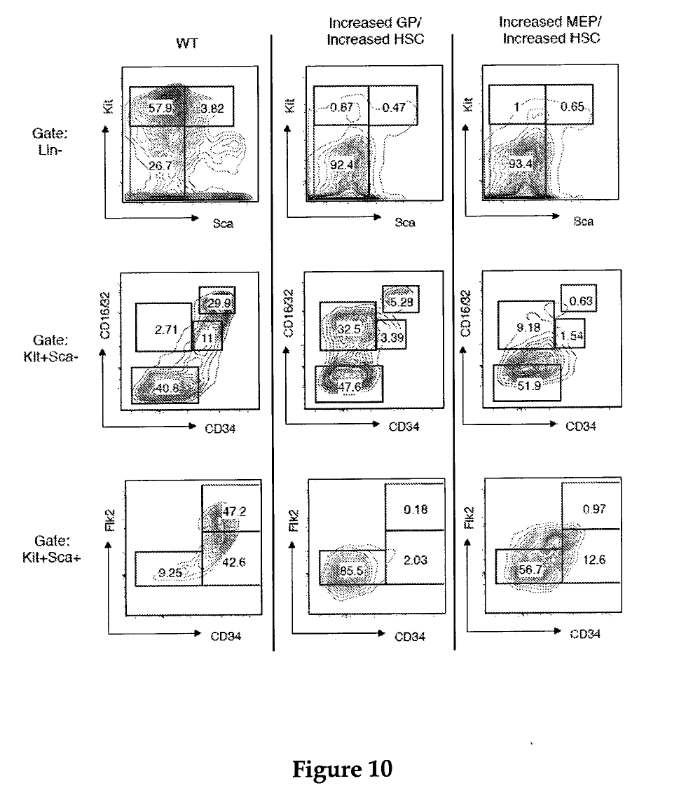

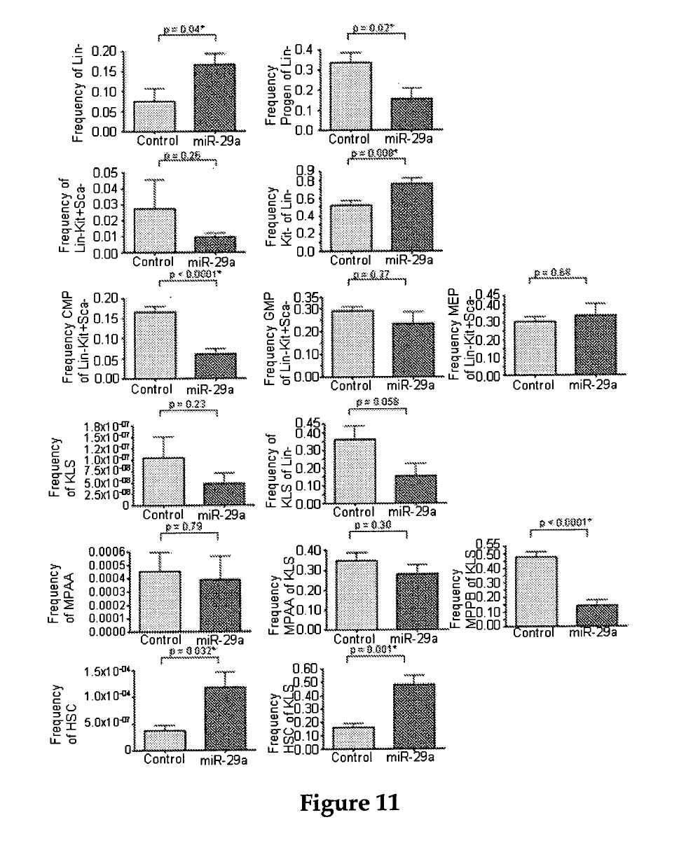

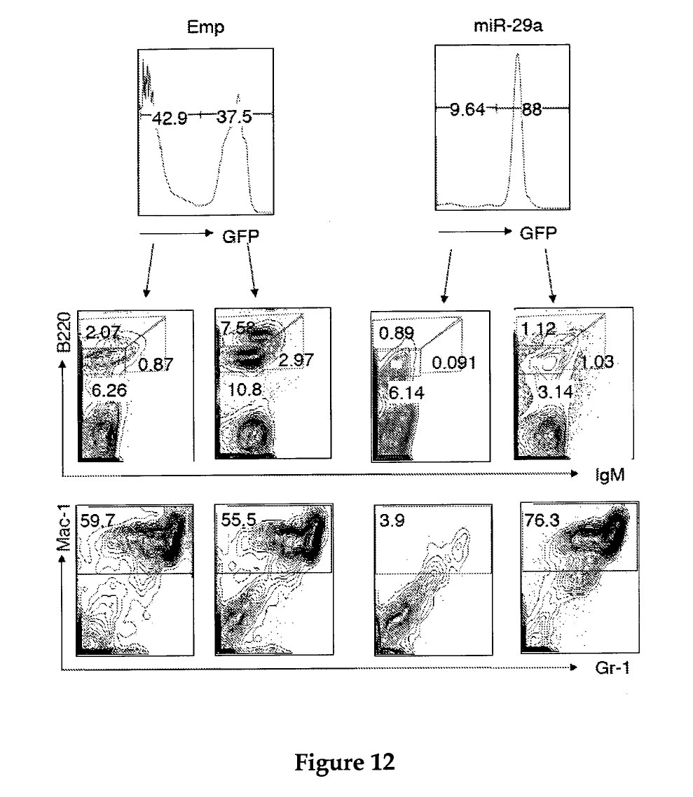

miR-29a-induced MPD may result from population expansion at the myeloid progenitor stage or proliferation of more mature myeloid cells. To distinguish between these possibilities, various stages of myeloid cells in the bone marrow were analyzed by flow cytometry. The analyses revealed that myeloid progenitors consistently exhibited changes in progenitor composition with variable increases in the frequency of GMP, MEP, CMP, and/or a novel myeloid progenitor population (FIG. 2C, FIG. 10). Analysis of myeloid cells in miR-29a chimeric mice revealed statistically significant left-shifted myeloid maturation, including higher absolute numbers of Lin⁻ cells (2.2 fold) and Lin⁻Kit⁻ cells (1.5 fold). The most immature hematopoietic compartment (Lin⁻Sca⁺Kit⁺, LSK) was not expanded in absolute numbers, although immunophenotypic long-term HSC were expanded in absolute numbers (3.1 fold increase) (FIG. 11). There was a marked decrease in Flk2⁺ LSK progenitors (2.5 fold) and B cell progenitors in the bone marrow and spleen. Consistent with the reduced B-lymphoid progenitors, mature B cells were decreased in the peripheral blood, bone marrow, and spleen (FIGS. 9 and 12). The MPD phenotype was reproducibly observed in 5 independent sets of mice, each containing 3-5 mice transduced with different miR-29a retroviral preparations. Together, these data indicate that over-expression of miR-29a expression significantly affects hematopoietic development by altering myeloid progenitor composition and promoting myeloid differentiation.

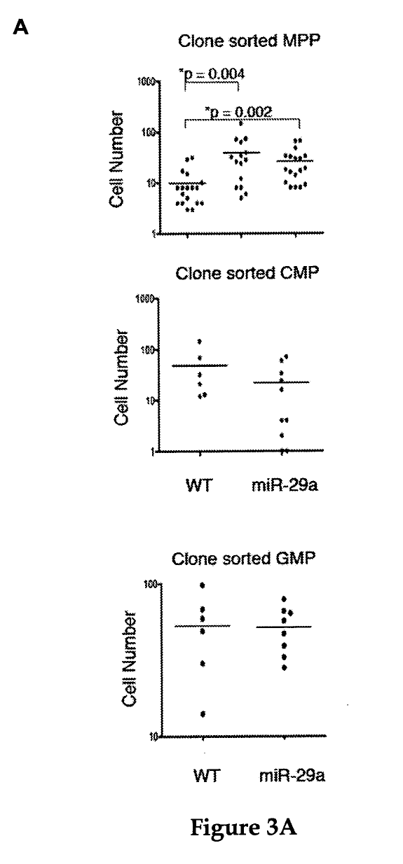

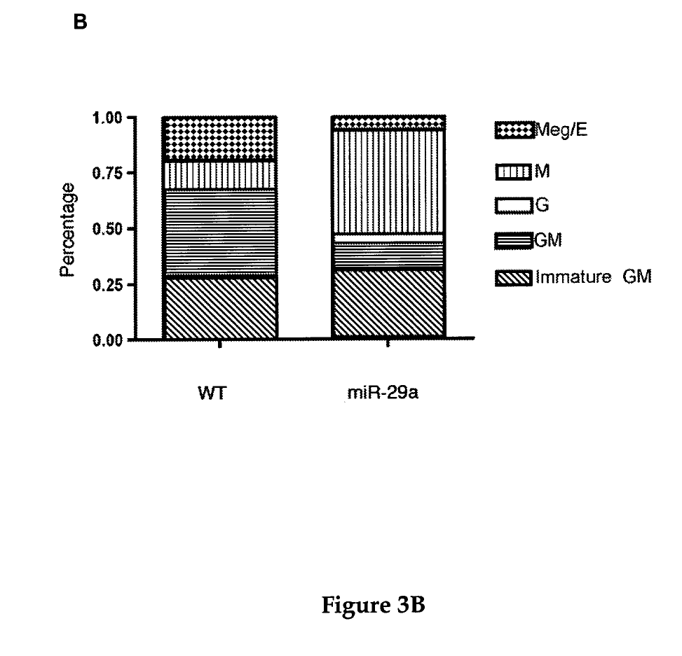

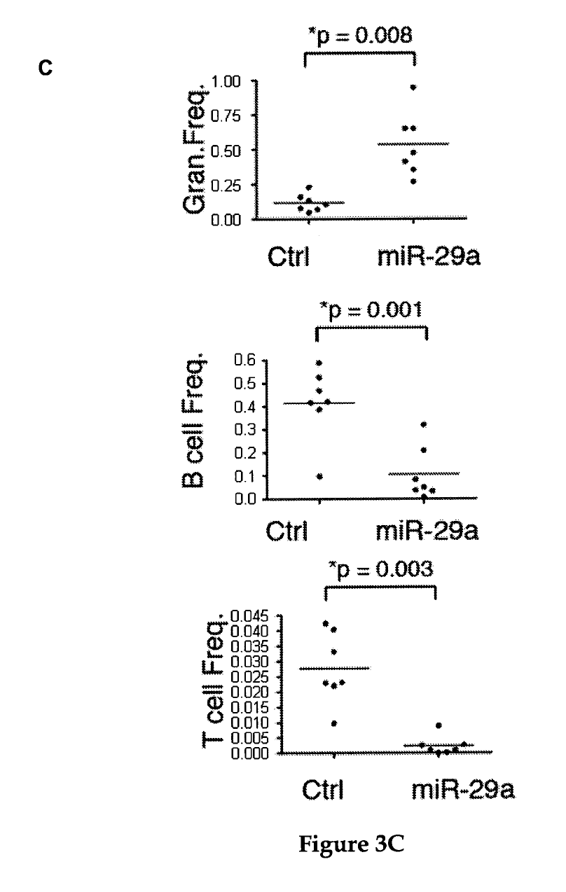

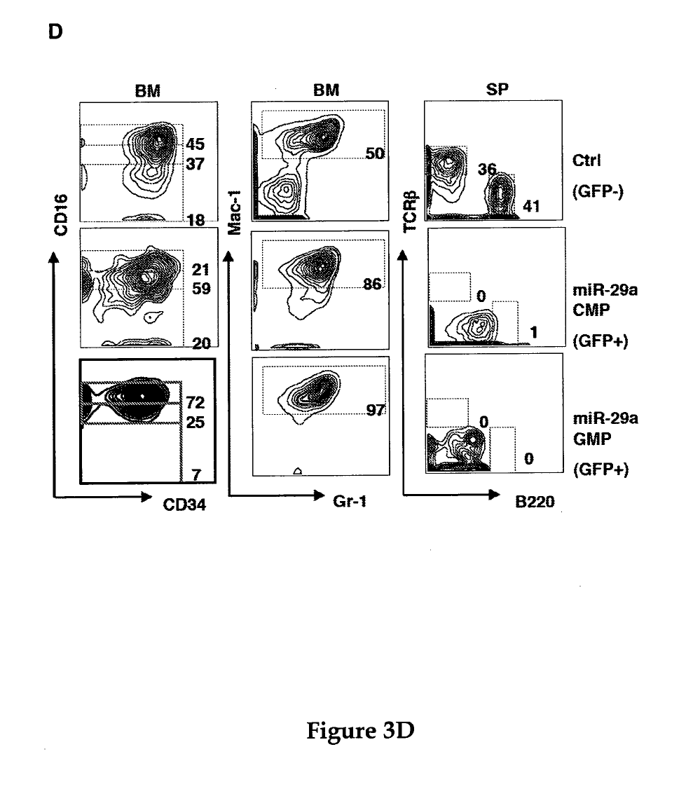

In order to assess the stage of hematopoiesis at which miR-29a exerts its pro-myeloid differentiation and proliferative effects, retrovirus infected (GFP⁺) HSC, MPP, CMP, and GMP were sorted from miR-29a-expressing chimeric mice and assessed the ability of single, sorted progenitors to proliferate and differentiate in vitro using liquid cultures. miR-29a MPP (Lin⁻Kit⁺Sca⁺CD34⁺FLK2⁻) exhibited an increased (˜2 fold) proliferative capacity relative to wild type MPP (p<0.005). Neither miR-29a-expressing GMP nor CMP exhibited significant differences in proliferative capacity compared to their wild type counterparts (FIG. 3A). Morphologic evaluation of cytospin preparations from the single-cell liquid cultures revealed that miR-29a-expressing MPP exhibited a relatively decreased capacity to form megakaryocyte-containing colonies and a relative increase in monocyte/macrophage colonies (FIG. 3B). These data show that over-expression of miR-29a acts at the level of the MPP to promote proliferation as well as establish a granulocyte/macrophage lineage bias among myeloid progenitors.

### Example 5

### miR-29a Over-Expression Converts Non-Self-Renewal Myeloid Progenitors Into Self-Renewal Populations

In normal hematopoiesis, HSC are the only cells capable of lympho-myeloid differentiation as well as long-term self-renewal. Aberrant acquisition of self-renewal capacity is likely an important step in myeloid leukemogenesis (Passegue et al., 2003). Analysis was performed to determine whether miR-29a-overexpressing non-self-renewing progenitors acquire this capacity during the early phase of MPD. MPP, CMP, and GMP were sorted from control (wild type or empty retrovirus transduced chimeric mice) or miR-29a-overexpressing primary chimeric mice before MPD development (approximately one month after the transfer) and transplanted purified progenitors (2−5×10³) into lethally irradiated recipients together with a radioprotective dose of wildtype bone marrow mononuclear cells. The bone marrows and spleens of transplanted mice were analyzed for donor derived cells by flow cytometry 2-4 months later, a time point at which normal progenitors and their progeny are no longer detectable in the peripheral blood or bone marrow due to the absence of self-renewal (Christensen and Weissman, 2001).

Mice receiving wild type MPP, CMP, and GMP possessed no detectable donor-derived cells (n=13). 66% (8/12) of mice transplanted with the miR-29a-over-expressing MPP, 75% (3/4) of mice transplanted with miR-29a-over-expressing CMP, and 75% (3/4) of mice transplanted with miR-29a-over-expressing GMP contained significant numbers of donor derived cells. The engrafted MPP cells exhibited a strong myeloid lineage bias with limited lymphoid output, similar to that found in the donor mice (FIG. 3C). Although phenotypic MPP were not detectable in the recipients that received miR-29a over-expressing MPP 4 months after the transplantation, CMP and/or GMP were persistent in these mice at least up to 6 months.

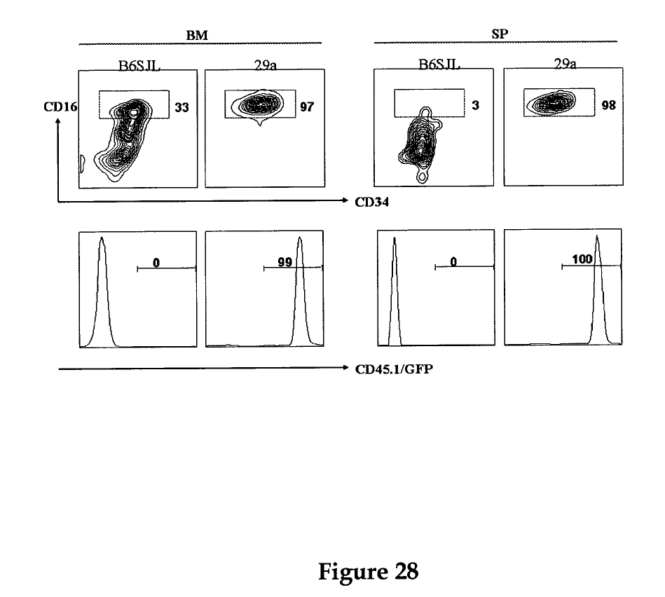

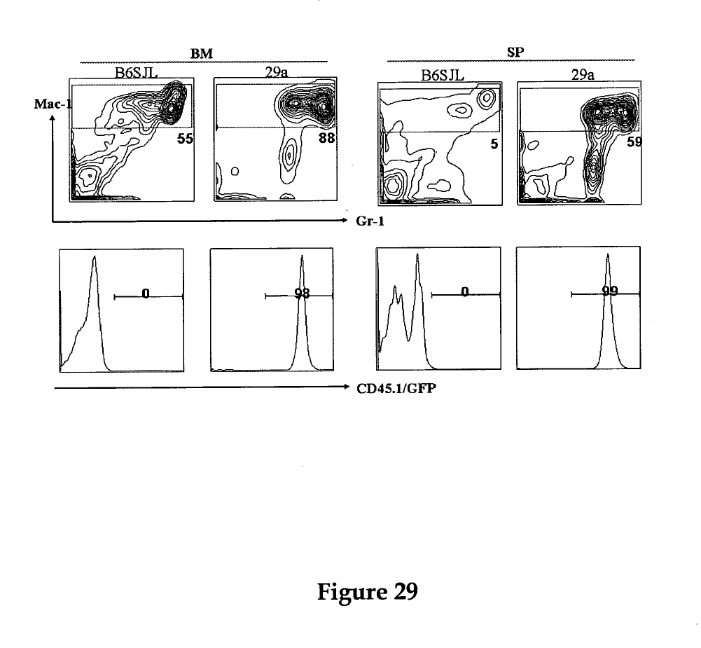

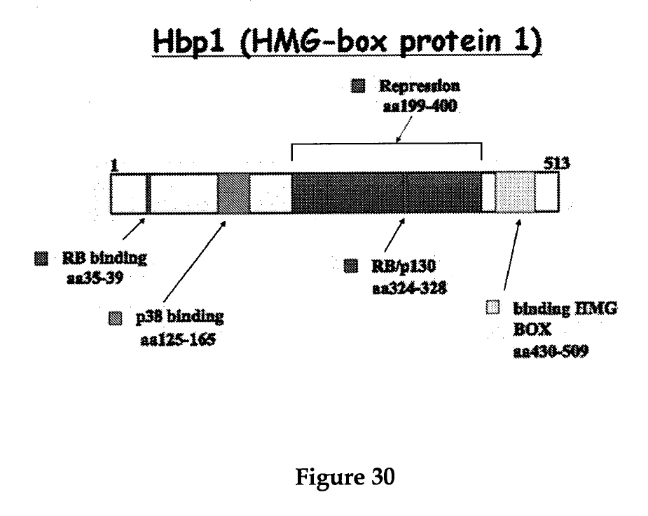

Mice transplanted with miR-29a-expressing CMP or GMP possessed donor-derived CMP and GMP, respectively, as well as their differentiated GFP+progeny (Mac1⁺Gr-1⁺ cells), even 2 months after the transplantation (FIG. 3D). No donor derived B or T cells were detected in the recipients, indicating that the observed myeloid progenitors were not generated by contaminating transplanted HSC (FIG. 3D). These results show that miR-29a induces aberrant self-renewal of CMP and GMP without impairing their normal ability to differentiate into monocytes or granulocytes (FIGS. 28 and 29).

### Example 6

### Enforced miR-29a Expression Leads to a High Incidence of Acute Myeloid Leukemia

Aberrant microRNA expression has been documented in both solid and hematopoietic malignancies (Garzon et al., 2006; Lu et al., 2005); however, there is no functional evidence that miRNAs induce leukemias originating from immature hematopoietic cells. Abnormal acquisition of self-renewal capability by myeloid progenitors and development of MPD in miR-29a chimeric mice suggests a pre-leukemic state of myeloid progenitors that may function as tumor-initiating cells and progress to acute leukemia. To test this possibility, bulk splenic or bone marrow cells were transferred from the chimeric mice into syngenic recipient mice.

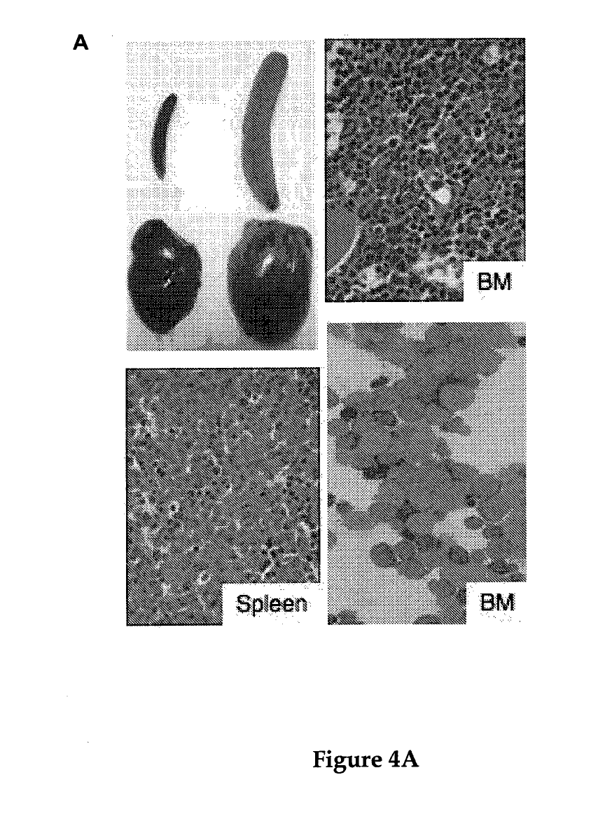

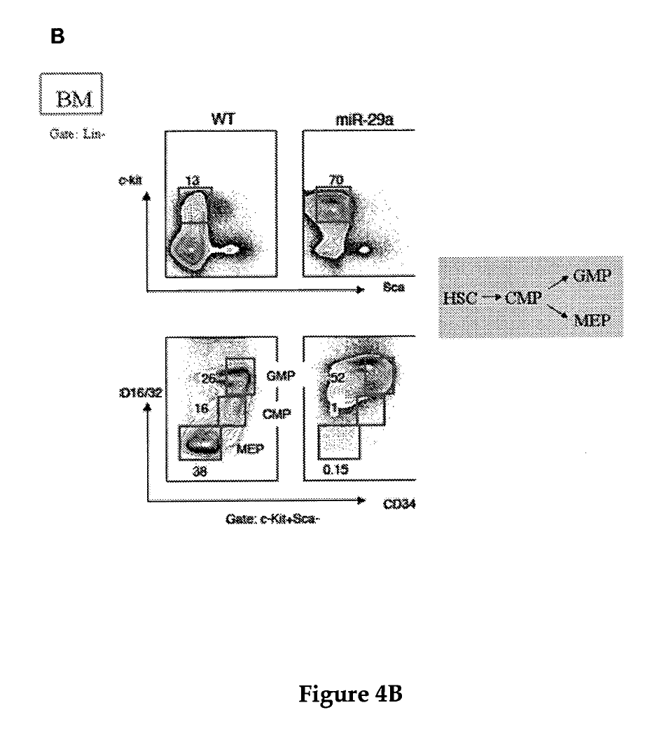

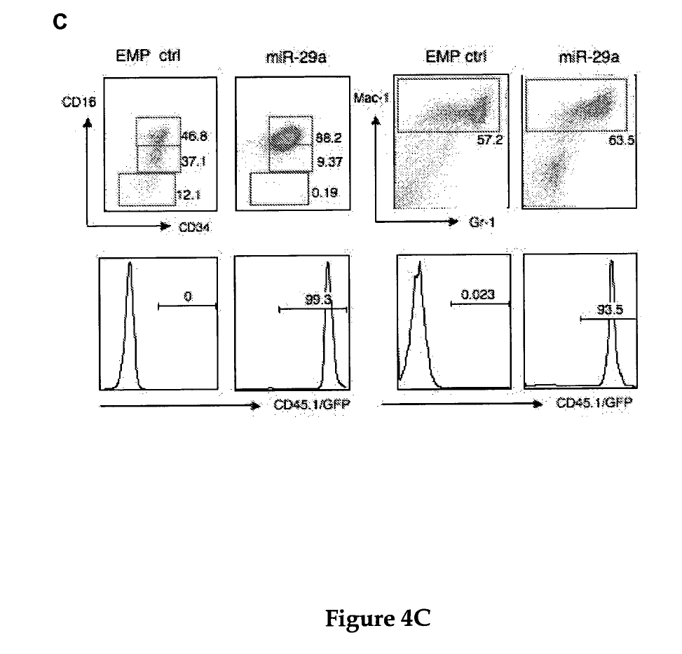

Almost all secondary recipients became moribund about 4 to about 8 months post-transplantation. At necropsy, these recipients exhibited profound hepatosplenomegaly. Histologic sections showed effacement of normal splenic and bone marrow architecture by an immature mononuclear population, consistent with leukemic blasts. Morphologic evaluation of cytospin preparations of total splenocytes and bone marrow cells from leukemic mice revealed increased numbers of myeloid blasts (range 25-80%), a phenotype consistent with progression to acute myeloid leukemia (AML) (FIG. 4A). Flow cytometric analysis of splenocytes or bone marrow cells showed increased numbers of Lin⁻Kit⁺Sca⁻ blast cells.

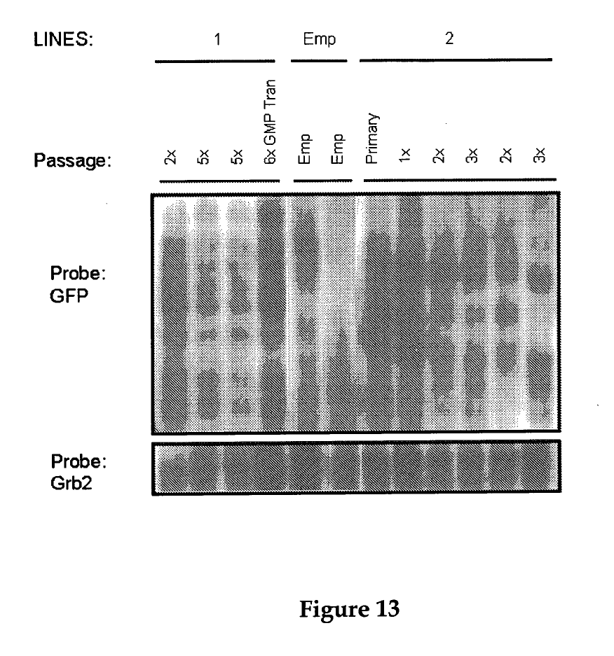

These blast populations typically showed a GMP-like (GMP-L), CMP-like (CMP-L) or a previously uncharacterized myeloid progenitor phenotype within the normal Lin⁻Kit⁺Sea⁻ progenitor compartment (FIG. 4B). Using total splenocytes or bone marrow mononuclear cells, the leukemia was serially transplantable up to 6 times over a period of 18 months. Southern blot analysis of retroviral integration sites using genomic DNA isolated from serially transplanted leukemia showed that the leukemia was likely oligoclonal, with no significant selection for particular clones during later passages (FIG. 13). Overall, these data show that the appearance of self-renewing miR-29a-expressing myeloid progenitor cells proceeds MPD disease progression to AML, indicating that aberrant acquisition of self-renewal by myeloid progenitors can be an early event during myeloid leukemogenesis.

### Example 7

### miR-29a Over-Expressing Myeloid Progenitors are Functional Leukemia Stem Cells

Human AML is organized hierarchically, similar to the normal hematopoietic system, and often possesses a leukemia stem cell (LSC) population capable of self-renewal and differentiation (Bonnet and Dick, 1997; Lapidot et al., 1994). Because miR-29a-induced AML exhibited serial transplantability, miR-29a-induced AML was examined to determine whether it contained a LSC population. As bone marrows and spleens of miR-29a-induced AML mice contained increased numbers of immunophenotypic GMP-L as well as more mature Mac-1⁺ cells, these two populations were sorted from leukemic mice to >99% purity and transplanted them into unirradiated recipients. Following transplantation, recipient mice were monitored for engraftment (Table 3).

TABLE 3
Summary of GMP-L transplants. FACS-purified GMP-L were transplanted
into non-irradiated or irradiated mouse recipients and monitored for
leukemia development for up to 8 months. Engraftment was assessed in
the peripheral blood or in the spleen and bone marrow of long-term
engrafted mice (>16 weeks) or moribund mice. Positive engraftment for
leukemia was determined by flow cytometry as well as morphologic
evaluation of cytospin preparations of total BM cells and/or splenocytes.

| # GMP-L cells | Number of mice |
|---|---|
| transplanted | engrafted |
| 2 | 0/3 |
| 20 | 5/8 |
| 200 | 8/13 |
| 2,000 | 5/5 |
| 2,0000 | 5/5 |

Sorted GMP-L stably engrafted recipients within 2 months after the transfer, and serial dilution experiments demonstrated that as few as 20 GMP-L cells could serially transplant the disease and give rise to an expanded GMP-L cell population without evidence of HSC or other normal myeloid progenitor contamination (Table 4).

TABLE 4
Selected patient characteristics for AML samples used in this study.

| | | | % | | FLT3- |
|---|---|---|---|---|---|
| | FAB | WHO | CD34 | | ITD |
| Sample | Category | Classification | blasts | Karyotype | status |
| AML1 | M0/M1 | AML, NOS | 95 | 46, XY[20] | N/A |
| AML2 | M5b | AML, NOS | 94 | 46, XX[20] | Neg. |
| AML3 | M5 | AML, NOS | 86 | 46, XY[20] | Neg. |
| AML4 | M1 | AML/MLD | 93 | 46, XY[20] | Neg. |
| AML8 | M2 | AML, NOS | 71 | 46, XY[20] | Neg. |
| AML10 | M2 | AML, NOS | 73 | 46, XY[20] | Neg. |
| AML11 | M4 | AML/MLD | 35 | 46, XY[20] | Neg. |
| AML12 | M4/M5 | AML from MDS | 24 | 46, XX[20] | Neg. |
| AML13 | M2/M4 | Therapy-realted | 32 | 46, XX[21] | N/A |
| | | AML | | | |
| AML14 | M5 | AML from | 24 | 46, XY[20] | Neg. |
| | | CMML | | | |
| AML15 | M1 | AML from MPD | 99 | 46, XX[20] | N/A |
| AML16 | M2 | AML with inv16 | 28 | inv16 | N/A |

FAB—French-American British,
NOS—not otherwise specified,
WHO—World Health Organization,
ITD—internal tandem duplication,
MLD—multilineage dysplasia,
MDS—myelodysplastic syndrome,
CMML—chronic myelomonocytic leukemia,
MPD—myeloproliferative disorder,
na—not available.

These GMP-L cells not only were capable of self-renewal but also gave rise to more mature monocytes/macrophages and granulocytes (FIG. 4C). In contrast, mice that received up to 5×10⁴ purified GFP⁺ Mac-1⁺/Mac-1⁺ Gr-1⁺ cells failed to engraft AML during an 8-months survey period. A similar situation was also observed with miR-29a over-expressing CMP-L cells. These results show that miR-29a transformed GMP-L cells are at least highly enriched for cells exhibiting the ability to self-renew and initiate a leukemic developmental hierarchy, consistent with LSC/leukemia initiating cell activity.

### Example 8

### A High Percentage of Human Acute Myeloid Leukemia Also Over-Express miR-29a

Analysis was performed to determine if miR-29a dependent induction of AML can occur. miR-29a expression was measured in FACS-purified LSC (Lin⁻CD34⁺CD38⁻) and non-LSC (Lin⁻CD34⁺CD38⁺) blasts from 12 diagnostic AML patient samples and compared miR-29a expression in AML LSC and non-LSC blasts to normal human HSC/progenitors, the normal counterpart to LSC blasts (Majeti et al., 2007a). miR-29a expression was higher (3-4 folds, p<0.01) in all AML as compared to normal committed myeloid progenitors, however, comparable to that in normal human HSC (FIGS. 1A, 1B). These results show that miR-29a expression is enhanced in human AML as compared to normal committed progenitors and indicate that miR-29a can play an important role in human myeloid leukemogenesis.

### Example 9

### miR-29a Regulates the G1 to S phase of Cell Cycle

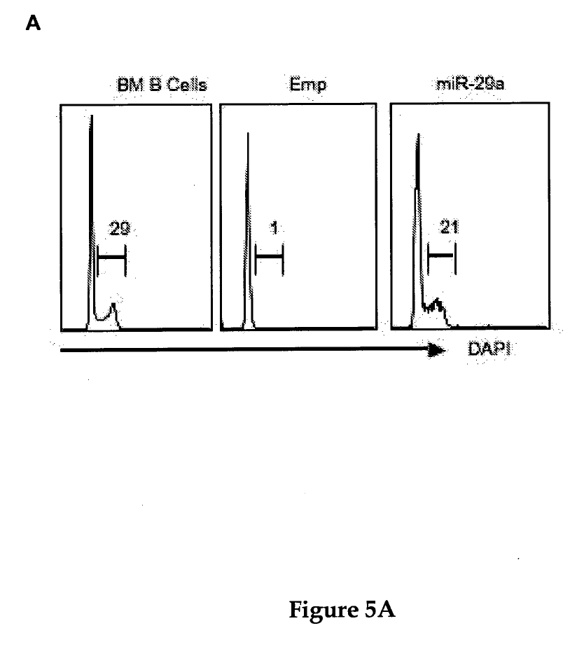

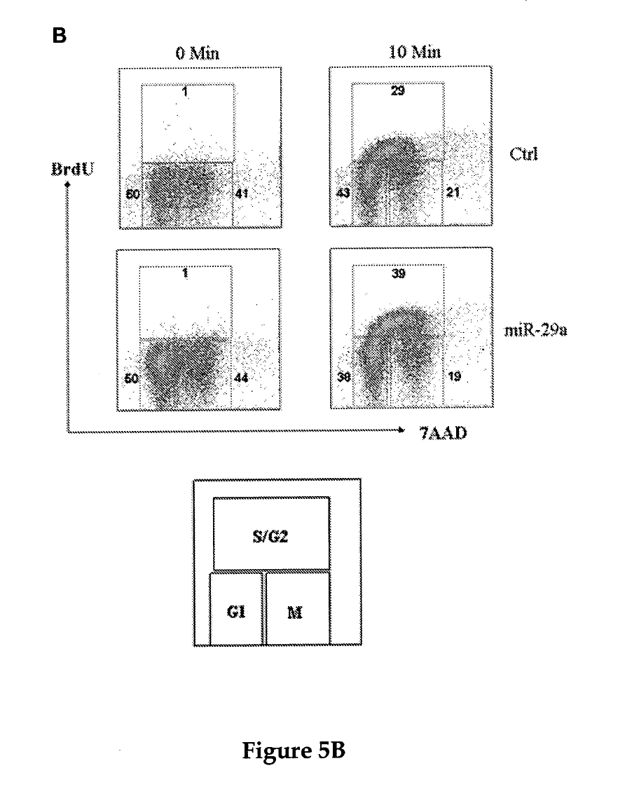

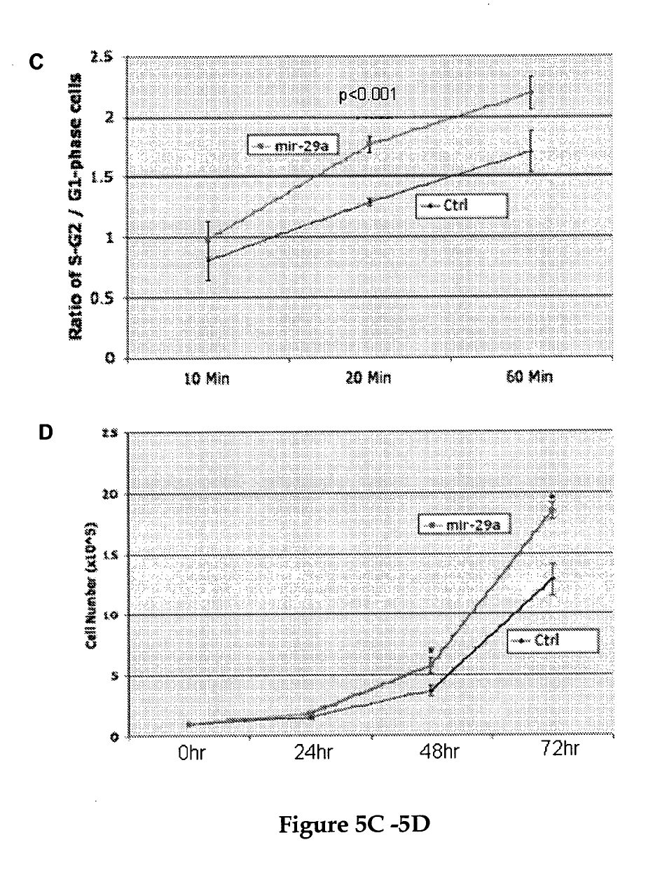

Development of myeloid leukemia in miR-29a primary chimeric mice indicates that miR-29a promotes proliferation or survival of myeloid cells. To distinguish between these two possibilities, the cell cycle status of myeloid cells in MPD mice was analyzed by flow cytometry. DAPI staining of splenocytes revealed that while wild type Mac-1⁺ Gr-1⁺ cells were not proliferating, approximately 20% of miR-29a-expressing cells contained greater than 2n DNA content, indicating active cell cycling (FIG. 5A).

To determine whether miR-29a positively regulates cell cycle progression, the G1 to S/G2 phase transition between wild type 293T and 293T cells over-expressing miR-29a was compared by in vitro BrdU incorporation assays. miR-29a over-expressing 293T cells entered S/G2 phase more rapidly than wild type 293T cells, with approximately 30% more cells entering cell cycle after 60 min of BrdU labeling (FIGS. 5B, 5C). The proliferation rate of miR-29a over-expressing 293T cells was significantly enhanced as compared to the wild type cells (FIG. 5C). There was no apparent effect of miR-29a over-expression on basal levels of apoptosis or apoptosis induced by serum starvation, indicating that ectopic expression of miR-29a does not exhibit a significant impact on cell survival.

### Example 10

### miR-29a Regulation Can Require Coordination of Multiple Target Genes

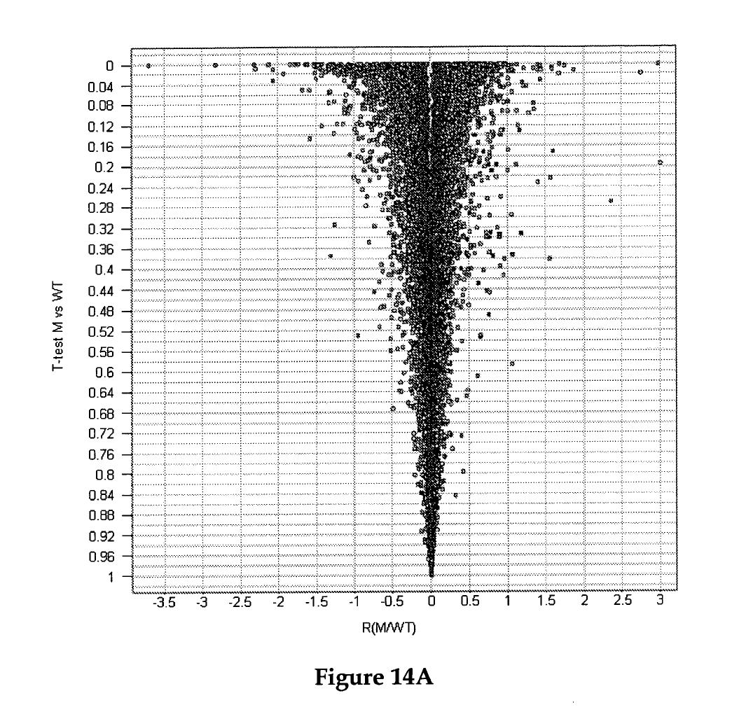

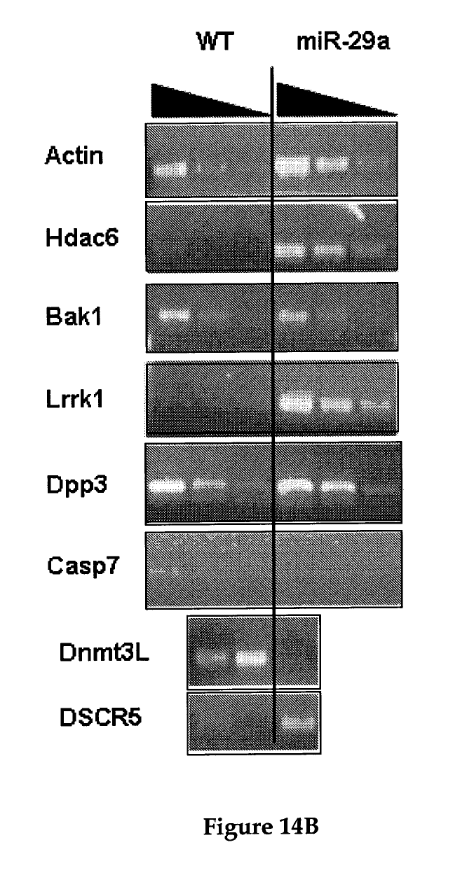

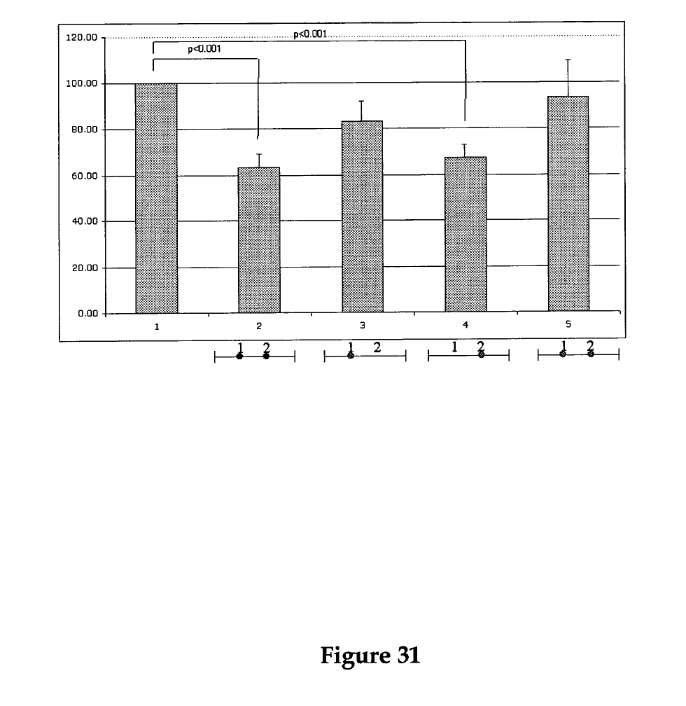

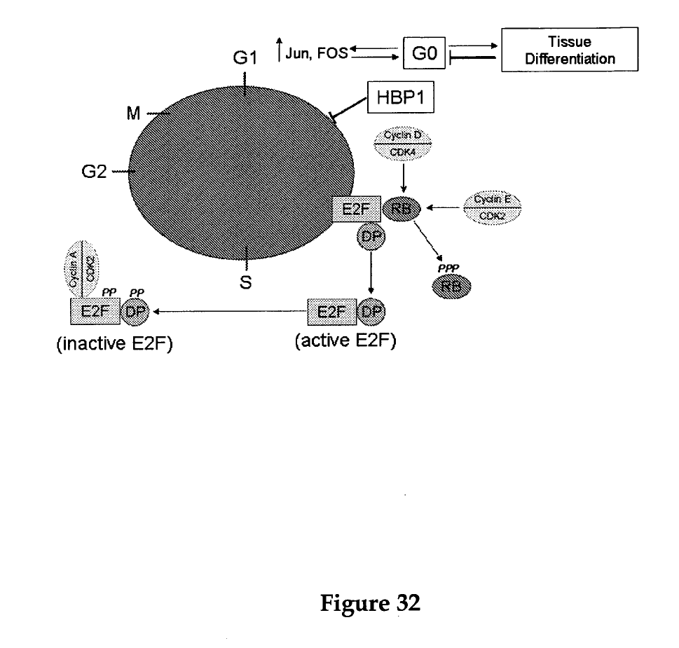

miRNAs can exert their biologic effect by regulating mRNA degradation and/or protein translation. To identify direct and indirect mRNA targets of miR-29a, mRNA expression in sorted Mac-1⁺ Gr-1⁺ cells from WT mice and miR-29a over-expressing primary chimeras was compared (FIG. 14). Filtering genes predicted to be targets of miR-29a (with a cut-off line of at least 2-fold decrease) was used to identify several tumor suppressors and cell cycle regulators that were significantly decreased in miR-29a granulocytes compared to wild type (Table 5 and Table 6). These genes included Slfn4, Dnmt3L, Cdc42ep2, and HBP1 (FIG. 31). HBP1 is a negative regulator of cell cycle progression at the G1 to S/G2 phase transition (Tevosian et al., 1997) (FIG. 32).

TABLE 5
Summary of genes most differentially down-regulated in miR-29a expressing granulocytes
(Mac-1⁺Gr-1⁺) versus WT granulocytes. Based on the fold differences
(more than 1.5) and p-value (<0.005), 30 genes were found to be downregulated while
10 genes were upregulated (shown in the box above). Among the 40 genes with altered
expression, 15 are known to related to either leukemia, apoptosis, cell proliferation and
differentiation or tumor suppression.

| | | | | Cell | Cell |
|---|---|---|---|---|---|
| | Tumor | | | Proliferation/Growth/ | Differentiation/ |
| | Suppressor | Leukemia | Apoptosis | Cytoskeleton | Commitment |
| Down | Dnmt31 (−1.5) | | Casp 7 (−0.2) | Cdc42ep2 (−2.3) | Dpp3 (−0.7) |
| Regulated | Pten (−0.6) | | | Ptpn7 (−1.5) | IL-8Ra (−2.8) |
| | Hbp1 (−0.6) | | | Slfn4 (−1.7) | |
| | | | Bak1 (−0.3) | | |
| Up | | DSCR5 (+1) | | Hdac6 (+1.5) | |
| Regulated | | | | Lrrk1 (+0.8) | |
| | | | | Ptprv (+1.6) | |
| | | | | Ppm1f (+0.6) | |

TABLE 6
Among the 40 genes with altered expression, 15 are known to related
to either leukemia, apoptosis, cell proliferation and differentiation or
tumor suppression.

| | | | | Cell |
|---|---|---|---|---|
| Tumor | | | Cell/Proliferation/ | Differentiation/ |
| Suppressor | Leukemia | Apoptosis | Growth | Commitment |
| 3 | 1 | 2 | 7 | 2 |

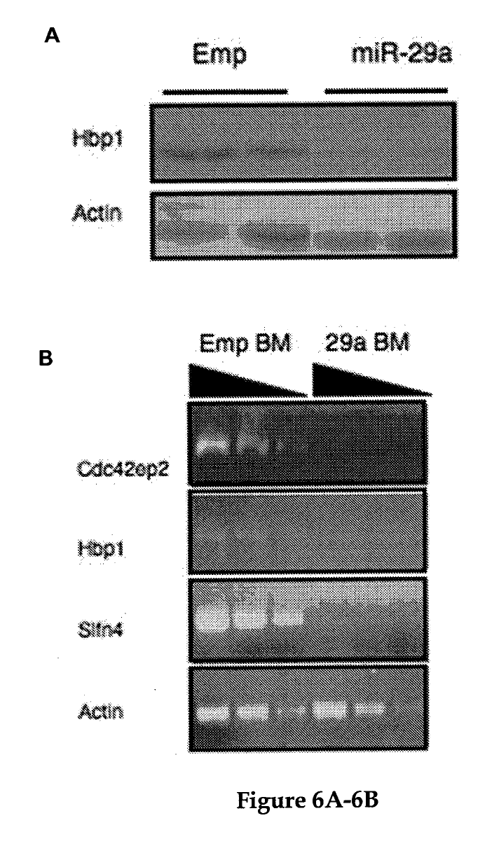

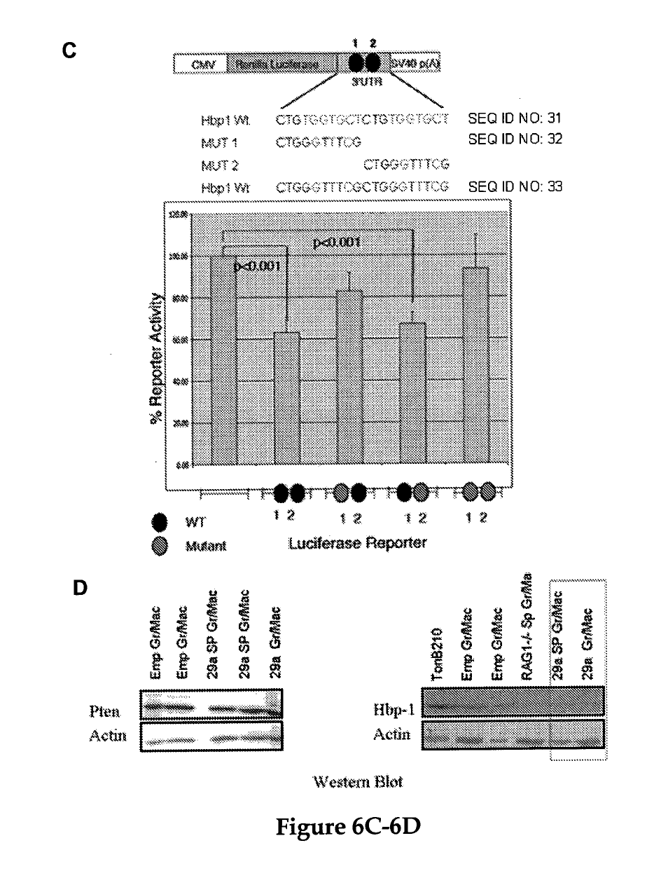

Consistent with being physiologic targets of miR-29a, decreased expression was confirmed for a subset of the genes by Western blot or semi-quantitative PCR analyses, including Cdc42ep2, Slfn4, and HBP1 (FIGS. 6A, 6B; FIG. 14B). Among the predicted targets, HBP1 was further investigated because of its previously described anti-proliferative activity and association with breast cancer invasiveness (Paulson et al., 2007; Tevosian et al., 1997). The mRNA encoding HBP1 contains two putative miR-29a binding sites within its 3′UTR (Targetscan v4.1; (Crimson et al., 2007; Lewis et al., 2005; Lewis et al., 2003).

In order to evaluate whether Hbp1 is an authentic target of miR-29a, luciferase reporter fusion genes were generated containing the Renilla luciferase coding gene and either the wild type or mutated 3′UTR of Hbp1. Fusion of the wild type 3′UTR of Hbp1 to the luciferase reporter attenuated luciferase activity, but a mutant 3′UTR of Hbp1 with mutation of either of the two putative miR-29a binding sites reversed the inhibitory effect. Mutation of both sites resulted in complete abrogation of the miR-29a-mediated inhibitory effect, indicating that the predicted miR-29a binding sites are imporant for mediating miR-29a inhibition of HBP1 expression (FIG. 6C).

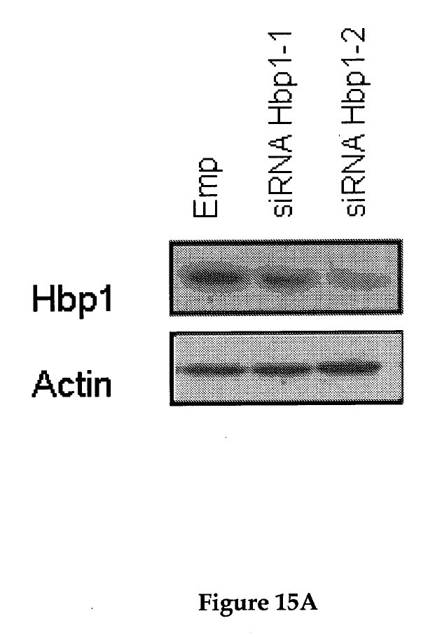

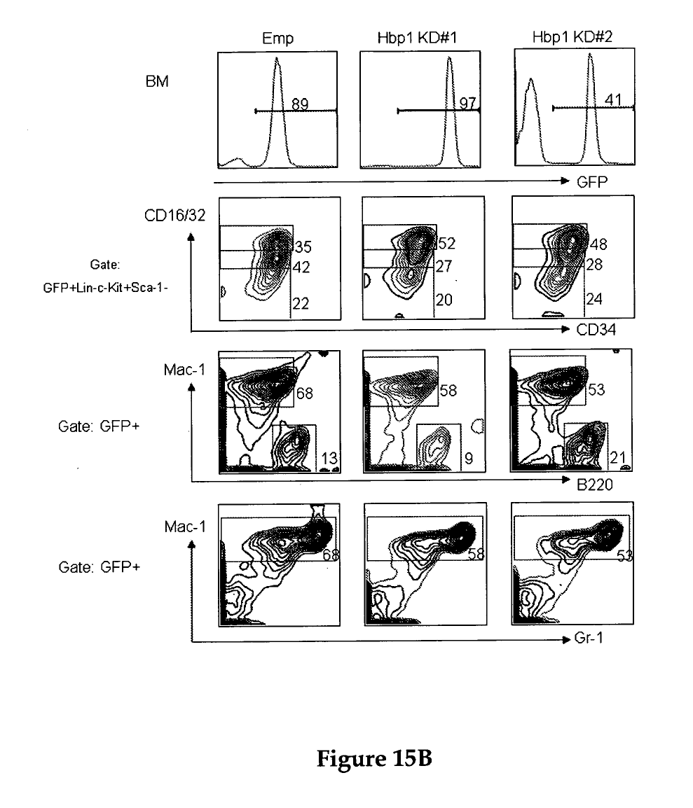

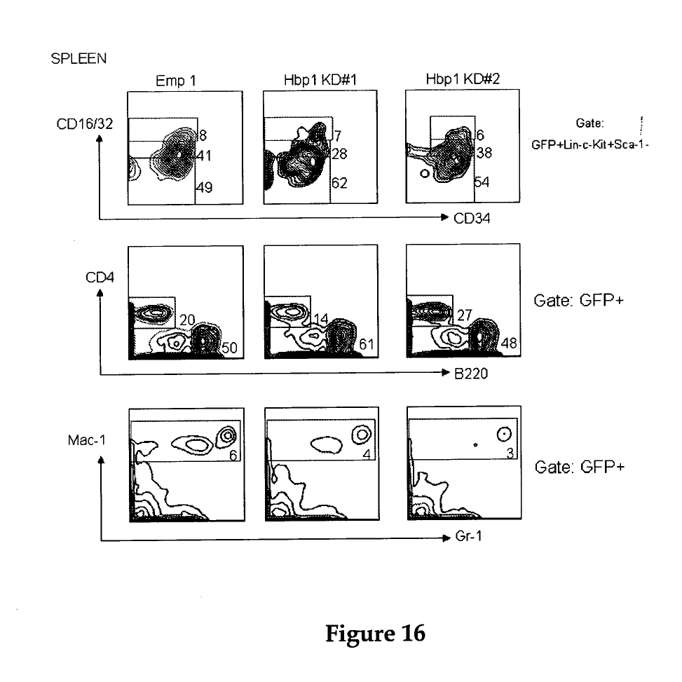

To determine whether repression of HBP1 in hematopoietic cells is sufficient to recapitulate miR-29a′s effects in vivo, an shRNA knock-down retroviral construct for Hbp1 was generated. Although this approach knocked-down HBP1 expression in hematopoietic cells, the same phenotypes as that with miR-29a over-expression was not observed (FIGS. 15 and 16). These results show that suppression of HBP1 alone is not sufficient and miR-29a can inhibit other genes that act in concert with, or independent of, Hbp1 in order to mediate its myeloproliferative and leukemogenic activity.

### Example 11

### Inhibition of miR-29a Reduces Leukemia Cell Proliferation

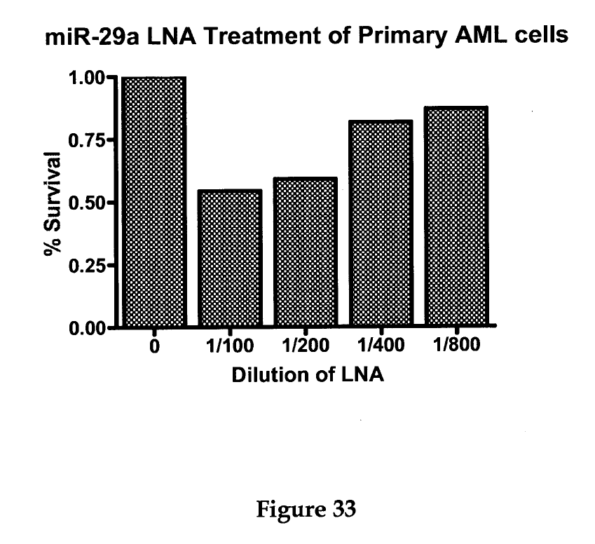

Human AML cells (5×10⁴) were seeded into a 96-well U-bottom plate with 100 μl Stemspan and cytokines (Flt3, TPO, SCF, IL-3, and IL-6). Cells were treated with mmu-miR-29a-ivp LNA (antagomir), having a sequence: 5′ ATTTCAGATGGTGCT 3′ (SEQ ID NO: 19). Survival was assessed by propidium iodide staining in a flow cytometer at 8 days after addition of LNA. Inhibition of miR-29a reduced AML cell proliferation in a dose-dependent manner (FIG. 33).

### Example 12

### Inhibition of miR-29a Treats MPD

As shown in Example 3, transgenic mice overexpressing miR-29a develop myeloproliferative disorder characterized by myeloid cell expansion and decreased B lymphocytes. An miR-29a antagomir, for example having the sequence of SEQ ID NO: 19, is introduced into mice with MPD. As described herein, introduction can be direct, such as through tail vein injection, or introduction can be via ex vivo manipulation and implantation of autologous cells, such as HSCs. The miR-29a antagomir is, for example, operably linked to a promoter in a retroviral-based vector. Splenocytes and bone marrow cells of treated mice are analyzed using flow cytometry. Mice treated with miR-29a antagomir show reduced myeloid lineage bias and increased production of B lymphocytes over untreated control mice with miR-29a-induced MPD.

### SUMMARY

While several miRNAs have been shown to regulate hematopoietic lineage fates and mature effector cells function, previous studies have not described their roles in the most immature hematopoietic cells, HSC and committed progenitors. The results described herein identify miR-29a as a promoter of myeloid differentiation and proliferation at the level of hematopoietic progenitors. The results descried herein also show that increased expression of miR-29a leads to aberrant acquisition of self-renewal capability by committed progenitors such as CMP and GMP. Since self-renewal capability is normally a feature of HSC, which also express the highest levels of miR-29a, the results described herein indicate that high miR-29a expression can contribute to HSC self-renewal. Acquisition of self-renewal capability by myeloid progenitors can have important clinical significance because abnormal acquisition of self-renewal capability by myeloid progenitors can represent a preleukemia stage in myeloid leukemogenesis. The extended life-span and proliferation of self-renewing progenitors may allow these cells to accumulate more genetic and/or epigenetic “hits” required to lead to full malignant transformation. The results described herein indicate that miRNAs can play important roles in the earliest stages of hematopoietic development and that misexpression of a single miRNA is sufficient to alter progenitor lineage specification, proliferation, and self-renewal. Notably, perturbations in all of these activities have been hypothesized to be required for the development of AML (Passegue et al., 2003).

The results presented herein also establish that a single miRNA can act as a robust oncogene. While miRNAs are postulated to act as tumor suppressors and oncogenes in human cancers (Calin and Croce, 2006), there is limited data establishing such roles for miRNAs using in vivo experimental models. The first oncomir, the miR-1792 cluster, was shown to decrease latency of lymphoma development in the c-myc transgenic setting (He et al., 2005); however, there is limited evidence that over-expression of a single miRNA contributes to cancer in a wild type genetic setting. miR-155 was shown to induce oligoclonal expansion of pre/proB cells when expressed as a B cell specific transgene (Costinean et al., 2006) and also induced a myeloproliferative disorder using experimental approaches similar to those described herein; however, in neither case did miR-155-induced disease progress to acute leukemia (O'Connell et al., 2008). In human AML, numerous miRNA profiling studies have shown that miRNA signatures can correlate with specific AML cytogenetic subgroups. While some miRNAs may serve as prognostic markers, such miRNA profiling studies have not shown a pathogenic role for miRNAs in AML (Garzon et al., 2008a; Garzon et al., 2008b; Marcucci et al., 2008). In this regard, the results described herein show that miR-29a is a miRNA that induces myeloid progenitor self-renewal which progress to AML.

These results also show the oncogenic role of miR-29a in vivo. Over-expression of miR-29a in HSC/progenitors initially causes self-renewal of myeloid progenitors and MPD-like disease. The disease then progresses to an AML that is organized hierarchically, as demonstrated by its ability to transfer the disease via a distinct LSC population. In a significant percentage of cases, aberrant acquisition of self-renewal in myeloid progenitors proceeds full leukemic transformation, indicating that self-renewal, while necessary for development of LSC, need not always represent the last step in leukemia development as has been suggested for some types of leukemia (Weissman, 2005). Taken together, these results demonstrate that altered expression of a miR-29a can induce leukemia, establishing a novel experimental model for studying AML initiation and progression. The presence of high miR-29a expression in human AML shows that miR-29a induced AML in mice is a relevant model for human AML and that miR-29a can serve as a potential target for AML therapies.

Self-renewal of AML stem cells has been previously linked to lipid phosphotase PTEN. The results shown herein indicate that PTEN protein is expressed at the normal levels in miR-29a induced AML as compared to WT (Mac-1⁺GR-1⁺) granulocyrtes/monocytes. Instead, expression of HBP1, Slfn4, Dnmt3L, and Cdc42ep2 was simultaneously reduced in the mutant cells as compared to WT controls. HBP1 was a target of miR-29a. HBP1 is a high mobility group protein and functions as repressor of G1 to G2/S phase cell cycling. Although knock-down of Hbp1 by siRNA did not recapitulate the phenotypes induced by miR-29a over-expression, a coordinating role of HBP1 with other factors such as Slfn4, Dnmt3L, and Cdc42ep2 in the disease development cannot be excluded. Recent experiments from Rajewsky's and Bartele's laboratories demonstrated that most miRNAs repress the expression of multiple proteins each with only moderate level of inhibition. miR-29a may regulate self-renewal of HSCs, LSCs, and myeloid progenitors through multiple targets including Hbp1.

Our results indicate that miR-29a plays an important role in the control of hematopoiesis and myeloid cell proliferation and leukemia. Human hematopoietic stem cells and myeloid leukemia and leukemia stem cells express a high levels of microRNA miR-29a. Animal studies indicated that expression of miR-29a alone in hematopoietic stem cells or myeloid lineage progenitors is sufficient to convert these stem cells or progenitors into myeloid leukemia stem cells, resulting in the development of acute myeloid leukemia. Over-expression of miR-29a positively regulates the development and cell-cycle progression of myeloid-lineage cells (Chronic Myeloid Leukemia (CML)-Like Disease). These results indicate that upregulation of miR-29a in myeloid cells can be a pre-leukemic state of myeloid leukemia.

Ectopic expression of miR-29a negatively regulates the development of B cells (Cell-fate determination). Furthermore, microarray analysis of miR-29a overexpressing bone marrow myeloid cells shows approximately 30 genes downregulated while 10 genes upregulated. Many of these genes may represent biologically relevant targets of miR-29a. One such target of miR-29a may be HMG-box Protein 1 (Hpb1).

These studies thus indicate that expression level of miR-29a may be used as a biomarker in the diagnosis of human myeloid leukemia. The expression of miR-29a in hematopoietic cells can be quantitatively determined by PCR or DNA chip assay. Therefore this invention also covers the methods to determine the level of miR-29a in hematopoietic cells, such as PCR, DNA and RNA microarrays to quantify miR-29a expression and siRNA microarrays can be designed as a tool for screening or diagnosis of myeloid leukemia in a subject having, or at risk of having a myeloid leukemia. In another aspect, the results described herein provide a method for treating a miR-29a expression-related myeloid leukemia.

In one embodiment, miR-29 inhibition can be used for the treatment of human AML using primary human AML samples in a xenotransplantation setting. In another embodiment, an siRNA chip-based assay may be used to diagnose human myeloid leukemia and as a therapeutic strategy to treat miR29a-related myeloid leukemia. This technology can be used for: detection, diagnosis and prognosis of human AML and other diseases of HSC/progenitors, absence/presence of miR-29a can be used to evaluate for residual and/or recurrent AML and inhibition of miR-29a expression can be a potential treatment for human AML and related diseases, either alone or in combination with conventional treatments. The methods of the invention can be used to identify other microRNAs involved in diseases of HSC/progenitors that are potential candidates for targeted therapy of diagnostic/prognostic tests.

### REFERENCES

Akashi, K., Traver, D., Miyamoto, T., and Weissman, I. L. (2000). A clonogenic common myeloid progenitor that gives rise to all myeloid lineages. Nature 404, 193-197.
Bonnet, D., and Dick, J. E. (1997). Human acute myeloid leukemia is organized as a hierarchy that originates from a primitive hematopoietic cell. Nat Med 3, 730-737.
Calin, G. A., and Croce, C. M. (2006). MicroRNA signatures in human cancers. Nat Rev Cancer 6, 857-866.
Chang, T. C., and Mendell, J. T. (2007). microRNAs in vertebrate physiology and human disease. Annu Rev Genomics Hum Genet 8, 215-239.
Chen, C., Ridzon, D. A., Broomer, A. J., Zhou, Z., Lee, D. H., Nguyen, J. T., Barbisin, M., Xu, N. L., Mahuvakar, V. R., Andersen, M. R., et al. (2005). Real-time quantification of microRNAs by stem-loop RT-PCR. Nucleic Acids Res 33, e179.
Chen, C. Z., Li, L., Lodish, H. F., and Bartel, D. P. (2004). MicroRNAs modulate hematopoietic lineage differentiation. Science 303, 83-86.
Chen, C. Z., and Lodish, H. F. (2005). MicroRNAs as regulators of mammalian hematopoiesis. Semin Immunol 17, 155-165.
Christensen, J. L., and Weissman, I. L. (2001). Flk-2 is a marker in hematopoietic stem cell differentiation: a simple method to isolate long-term stem cells. Proc Natl Acad Sci USA 98, 14541-14546.
Costinean, S., Zanesi, N., Pekarsky, Y., Tili, E., Volinia, S., Heerema, N., and Croce, C. M. (2006). Pre-B cell proliferation and lymphoblastic leukemia/high-grade lymphoma in E(mu)-miR155 transgenic mice. Proc Natl Acad Sci USA 103, 7024-7029.
Ebert, M. S., Neilson, J. R., Sharp, P. A. (2007). MicroRNA sponges: competitive inhibitors of small RNAs in mammalian cells. Nat. Methods 4, 721-726.
Garzon, R., and Croce, C. M. (2008). MicroRNAs in normal and malignant hematopoiesis. Curr Opin Hematol 15, 352-358.
Garzon, R., Fabbri, M., Cimmino, A., Calin, G. A., and Croce, C. M. (2006). MicroRNA expression and function in cancer. Trends Mol Med 12, 580-587.
Garzon, R., Garofalo, M., Martelli, M. P., Briesewitz, R., Wang, L., Fernandez-Cymering, C., Volinia, S., Liu, C. G., Schnittger, S., Haferlach, T., et al. (2008a). Distinctive microRNA signature of acute myeloid leukemia bearing cytoplasmic mutated nucleophosmin. Proc Natl Acad Sci USA 105, 3945-3950.
Garzon, R., Volinia, S., Liu, C. G., Femandez-Cymering, C., Palumbo, T., Pichiorri, F., Fabbri, M., Coombes, K., Alder, H., Nakamura, T., et al. (2008b). MicroRNA signatures associated with cytogenetics and prognosis in acute myeloid leukemia. Blood 111, 3183-3189.
Grimson, A., Farh, K. K., Johnston, W. K., Garrett-Engele, P., Lim, L. P., and Bartel, D. P. (2007). MicroRNA targeting specificity in mammals: determinants beyond seed pairing. Mol Cell 27, 91-105.
He, L., and Hannon, G. J. (2004). MicroRNAs: small RNAs with a big role in gene regulation. Nat Rev Genet 5, 522-531.
He, L., Thomson, J. M., Hemann, M. T., Hemando-Monge, E., Mu, D., Goodson, S., Powers, S., Cordon-Cardo, C., Lowe, S. W., Hannon, G. J., and Hammond, S. M. (2005). A microRNA polycistron as a potential human oncogene. Nature 435, 828-833.
Krützfeldt J, Rajewsky N, Braich R, Rajeev K G, Tuschl T, Manoharan M, Stoffel M. (December 2005). Silencing of microRNAs in vivo with ‘antagomirs’. Nature 438 (7068): 685-9.
Lapidot, T., Sirard, C., Vormoor, J., Murdoch, B., Hoang, T., Caceres-Cortes, J., Minden, M., Paterson, B., Caligiuri, M. A., and Dick, J. E. (1994). A cell initiating human acute myeloid leukaemia after transplantation into SCID mice. Nature 367, 645-648.
Lewis, B. P., Burge, C. B., and Bartel, D. P. (2005). Conserved seed pairing, often flanked by adenosines, indicates that thousands of human genes are microRNA targets. Cell 120, 15-20.
Lewis, B. P., Shih, I. H., Jones-Rhoades, M. W., Bartel, D. P., and Burge, C. B. (2003). Prediction of mammalian microRNA targets. Cell 115, 787-798.
Lu, J., Getz, G., Miska, E. A., Alvarez-Saavedra, E., Lamb, J., Peck, D., Sweet-Cordero, A., Ebert, B. L., Mak, R. H., Ferrando, A. A., et al. (2005). MicroRNA expression profiles classify human cancers. Nature 435, 834-838.
Majeti, R., Park, C. Y., and Weissman, I. L. (2007a). Identification of a Hierarchy of Multipotent Hematopoietic Progenitors in Human Cord Blood. Cell Stem Cell 1, 635-645.
Majeti, R., Park, C. Y., and Weissman, I. L. (2007b). Identification of a hierarchy of multipotent hematopoietic progenitors in human cord blood. Cell Stem Cell 1, 635-645.
Manz, M. G., Miyamoto, T., Akashi, K., and Weissman, I. L. (2002). Prospective isolation of human clonogenic common myeloid progenitors. Proc Natl Acad Sci USA 99, 11872-11877.
Marcucci, G., Radmacher, M. D., Maharry, K., Mrozek, K., Ruppert, A. S., Paschka, P., Vukosavljevic, T., Whitman, S. P., Baldus, C. D., Langer, C., et al. (2008). MicroRNA expression in cytogenetically normal acute myeloid leukemia. N Engl J Med 358, 1919-1928.
O'Connell, R. M., Rao, D. S., Chaudhuri, A. A., Boldin, M. P., Taganov, K. D., Nicoll, J., Paquette, R. L., and Baltimore, D. (2008). Sustained expression of microRNA-155 in hematopoietic stem cells causes a myeloproliferative disorder. J Exp Med 205, 585-594.
Passegue, E., Jamieson, C. H., Ailles, L. E., and Weissman, I. L. (2003). Normal and leukemic hematopoiesis: are leukemias a stem cell disorder or a reacquisition of stem cell characteristics? Proc Natl Acad Sci USA 100 Suppl 1, 11842-11849.
Paulson, K. E., Rieger-Christ, K., McDevitt, M. A., Kuperwasser, C., Kim, J., Unanue, V. E., Zhang, X., Hu, M., Ruthazer, R., Berasi, S. P., et al. (2007). Alterations of the HBP1 transcriptional repressor are associated with invasive breast cancer. Cancer Res 67, 6136-6145.
Tang, G., Xiang, Y., Kang, Z., Mendu, V. Tang, X., Jia, X., Chen, Q., Tang, X. (2008). Small RNA Technologies: siRNA, miRNA, antagomiR, Target Mimicry, miRNA Sponge and miRNA Profiling. Current Perspectives in microRNAs (miRNA), Ying, S-Y. (ed.), Springer Science and Business Media, 17-33.
Tevosian, S. G., Shih, H. H., Mendelson, K. G., Sheppard, K. A., Paulson, K. E., and Yee, A. S. (1997). HBP1: a HMG box transcriptional repressor that is targeted by the retinoblastoma family. Genes Dev 11, 383-396.
Weissman, I. (2005). Stem cell research: paths to cancer therapies and regenerative medicine. Jama 294, 1359-1366.
Yang, L., Bryder, D., Adolfsson, J., Nygren, J., Mansson, R., Sigvardsson, M., and Jacobsen, S. E. (2005). Identification of Lin(−)Scal(+)kit(+)CD34(+)F1t3-short-term hematopoietic stem cells capable of rapidly reconstituting and rescuing myeloablated transplant recipients. Blood 105, 2717-2723.

We claim:

1. A method for treating a miR-29a-induced myeloproliferative disorder in a subject, the method comprising administering to the subject an miRNA inhibitory nucleic acid specific to miR-29a.

2. The method of claim 1, wherein the myeloproliferative disorder is a leukemia.

3. The method of claim 2, wherein the leukemia is selected from the group consisting of acute myelogenous leukemia, chronic myelogenous leukemia, chronic neutrophilic leukemia, and chronic eosinophilic leukemia.

4. The method of claim 1, wherein the miRNA inhibitory nucleic acid is an antagomir.

5. The method of claim 4, wherein the antagomir has the nucleotide sequence of SEQ ID NO: 19.

6. A method for diagnosing a miR-29a-induced myeloproliferative disorder in a patient, the method comprising comparing miR-29 expression in a myeloid cell from the patient with miR-29a expression in a myeloid cell from a control subject, wherein greater miR-29 expression in the patient indicates a a miR-29a-induced myeloproliferative disorder.

7. The method of claim 6, wherein the myeloproliferative disorder is a leukemia.

8. The method of claim 7, wherein the leukemia is selected from the group consisting of acute myelogenous leukemia, chronic myelogenous leukemia, chronic neutrophilic leukemia, and chronic eosinophilic leukemia.

9. A method for reducing miR-29a-induced proliferation of a myeloid cell comprising administering to the myeloid cell an miRNA inhibitory nucleic acid specific to miR-29a.

10. The method of claim 9, wherein the myeloid cell is selected from the group consisting of a monocyte, a granulocyte, a mast cell, a basophil, and a megakaryocyte.

11. The method of claim 9, wherein the miRNA inhibitory nucleic acid is an antagomir.

12. The method of claim 11, wherein the antagomir has the sequence of SEQ ID NO: 19.

13. A method of identifying a compound for reducing miR-29a-induced myeloid cell proliferation, the method comprising:
(a) administering to a non-human test mammal a candidate compound, wherein the test mammal comprises myeloid cells overexpressing miR-29a; and
(b) comparing miR-29a expression in myeloid cells from the test mammal with miR-29a expression in myeloid cells from a non-human control mammal, wherein the control mammal overexpresses miR-29a;
wherein reduced miR-29a expression in the test mammal indicates that the candidate compound is a compound for reducing myeloid cell proliferation.

14. The method of claim 13, wherein the non-human mammal has a myeloproliferative disorder.

15. The method of claim 13, wherein the myeloid cells are selected from the group consisting of monocytes, granulocytes, mast cells, basophils, and megakaryocytes.
